(19) United States
(12) Patent Application Publication
(10) Pub. No.: US 2004/0166183 A1
(43) Pub. Date: Aug. 26, 2004

(54) Methods and means for preventing or treating inflammation or pruritis

(76) Inventors: Johanna G. H. Ruseler-van Embden, Berkenwoude (NL); Leonarda M.C. van Lieshout, Rotterdam (NL); Jon D. Laman, Amsterdam (NL)

Correspondence Address: TRASK BRITT, P.O. BOX 2550, SALT LAKE CITY, UT, 84110, US

(21) Appl. No.: 10/727,345

(22) Filed: Dec. 1, 2003

Related U.S. Application Data
(63) Continuation-in-part of application No. 09/716,612, filed on Nov. 20, 2000, now Pat. No. 6,723,354.
Application No. 09/716,612 is a continuation of application No. PCT/NL99/00312, filed on May 20, 1999.

(30) Foreign Application Priority Data
May 20, 1998 (EP) 98201694.1

(51) Int. Cl. A61K 35/78

(52) U.S. Cl. 424/773

(57) ABSTRACT

The invention relates to methods and means for preventing, treating or reducing inflammation by inhibiting proteolytic activity, more specifically for preventing or reducing inflammations of skin or intestine. The invention provides a method for reducing or preventing an inflammation comprising subjecting a mammal to a treatment with at least one inhibitor which is capable of inhibiting proteolytic activity. In a preferred embodiment of the invention, said inhibitor is a plant product, such as potato juice or an inhibitor derived thereof.

### CROSS-REFERENCE TO RELATED APPLICATION

This application is a continuation-in-part of U.S. application Ser. No. 9/716,612, filed Nov. 20, 2000 as a national filing from international patent application PCT/NL99/00312, filed May 20, 1999, designating the United States of America, the contents of the entirety of which is incorporated by this reference.

### TECHNICAL FIELD

The invention relates generally to methods and means for preventing, treating or reducing inflammation by inhibiting proteolytic activity, and more specifically for preventing or reducing inflammations of skin or intestine.

### BACKGROUND

Inflammations of skin (dermatitis) or intestine (enteritis) are of various origins. Initially, allergic reactions, infections with (pathogenic) micro organisms, excoriation by chemical or physical means, and other causes are instrumental in causing an inflammation. These causal events are immediately followed by the so necessary reaction of the body, resulting in an interplay of actions and events aiming at restoration of the skin or intestine in its original state. In this interplay of cause and effect, various activities of proteolytic enzymes are seen. Granulocytes, mast-cells, macrophages and other immediate actors in inflammatory responses and attracted by cytokines to a site of inflammation, contain (and secrete) proteases, such as chymotryptic protease and elastase, that act as mediators or are instrumental in cleaving and removing proteins derived from pathogens or from the surrounding degenerated tissue. Bacteria, either as primary causal agent, or during a secondary infection, and other (pathogenic) micro-organisms, secrete proteases that damage the surrounding tissue for their purposes. In this battlefield between host and invader, excess proteolytic reactions are kept at bay by, often very specific, protease inhibitors. Well known are proteinase/proteinase inhibitor systems such as PMN-elastase/alpha-1-proteinase inhibitor and cathepsin G/alpha-1-antichymotrypsin.

Proteolytic enzymes in themselves, however, can also be a cause of inflammation. This is especially the case for digestive enzymes, which are found in the intestinal tract. In order to degrade dietary protein, the stomach, the pancreas and the small intestinal brush border secrete several kinds of proteases. Pepsin from the stomach works optimal at pH 2, pancreatic and brush border enzymes, such as trypsin, chymotrypsin and elastase work optimal at pH 7-8. In adults, the small intestine has a length of seven meters and the transit time of its contents is about 3 hours; this part of the intestine is colonised by only a few bacteria but is filled with a watery mixture of food and a wide array and large quantities of digestive enzymes, such as lipases and proteases. However, in the large intestine (colon and caecum) the water content is greatly reduced, and the activity of the enzymes is neutralised by i.e. bacteria. Neutralised and digested remnants of food and bacteria (feces) finally leave the body via the rectum. Only when the colon cannot effectively reduce the water content and neutralise the enzymes, the feces may still contain proteolytic activity, which, during periods of diarrhoea or fecal incontinence, may be very irritating to intra-anal and perineal skin.

The skin, especially of humans, is, although it is protected by the stratum corneum which consists mainly of keratin, as any other proteinaceous substance, very susceptible to the proteolytic action of proteases, consequently fluid like small intestinal content may cause severe inflammation.

In babies and infants, the intestine is much less well developed, especially the colon functions different from that in adults. This is the reason why digestive enzymes in feces of babies and infants are not neutralized; the contents of feces resemble more the contents of the small intestine, albeit having passed the colon. Therefore, perineal (perianal) dermatitis is more often found with babies or infants than with adults. Also, (hospitalised) infants and children with gastrointestinal disorders are prone to such a dermatitis Such a dermatitis or prunitis, defined by itchiness skin erythema, vesicular, wetness, oedema or disruption (excoriation) of perineal skin, is also found with diaper rash, and can manifest itself in rather mild to very severe forms. With diaper rash, complicating factors are the accumulation of urine, whereby ureum is converted by fecal bacteria to ammonia, thereby raising the pH to an even better value for the activity of proteolytic enzymes. Since the skin is extremely susceptible to infections, care should be taken to prevent such inflammations related to (fecal) proteolytic activity.

Yet other cases of dermatitis are found with patients that have a stoma, e.g. as a result of resections of colon and/or ileum. Pouchitis, an intestinal inflammation, is a major complication of ileoanal anastomosis with reservoir construction after colon resection and is characterised by clinical symptoms and inflammation of the reservoir (pouch). Peristomal (circumstomal) dermatitis is found with those patients that have been provided with an ileostoma that opens up at the surface of the abdomen, ending in an artificial reservoir that needs to be emptied daily. In inflammatory bowel diseases (IBD, such as Crohn's disease (CD), ulcerative colitis (UC) and pouchitis), and inflammation with an unknown aetiology, the role of the intestinal flora and pathogens, proteolytic enzymes derived from these micro-organisms and endogenous (e.g. pancreatic or leukocyte/granulocyte) proteolytic enzymes and their contribution to degradation of protecting mucoglycoproteins and the underlying tissues is not understood.

Especially in above cases where the colon is removed or its function is affected or immature, it is evident that the proteolytic activity is still very high when the feces is excreted, leading to various degrees of perineal dermatitis.

It goes without saying that many medications and personal care items have been developed in order to remedy the (severely) itchy and often painful consequences of above discussed inflammations. General anti-inflammatory therapy often resorts to treatment with corticosteroids, despite the serious side-effects that are often seen with these medicaments. Other ways of treating are mainly based on providing either a protective layer to the skin, e.g by applying a lipid-based ointment, containing additives such as zinc, or by frequently cleaning an area at risk. Special personal care items have been developed, varying from specific wet wipes for perineal care, diapers that stay very dry despite heavy soiling by the child or patient, to products (stoma care appliances), such as adhesive and absorbing discs and stoma rinsing fluid, that are specifically designed for stoma care with patients with ilcostomy or ileo-anal anastomosis.

However, none of these treatments can really do no more than alleviate one or more of above and below described clinical symptoms.

The invention provides a method for treating, reducing or preventing an inflammation or pruritis comprising subjecting a mammal to a treatment with at least one inhibitor which is capable of inhibiting proteolytic activity. Preferably, the invention provides a method whereby a protease produced or secreted by for example granulocytes, mastcells, macrophages and other actors in inflammatory processes is inhibited. The invention is applicable to human and veterinary medicine and care.

A preferred embodiment of the invention is wherein said mammal is a human suffering from for example dermatitis or pruritis. Treating for example a dermatitis with a protease inhibitor reduces the proteolytic activity of the proteases involved in the inflammation pruritis. Especially when, in the interplay of causes and effects seen during inflammation, the activity of proteolytic enzymes is too high, the invention provides a method to reduce this activity (be it from host or from invader) by treatment with at least one inhibitor which is capable of inhibiting proteolytic activity.

Said treatment is provided by applying said inhibitor in an ointment, cream, gel, powder, or any other suitable form to the location of the inflammation. These substances can for example also be carried on wipes impregnated with an inhibitor, in sprays or in rinsing fluid.

In a preferred embodiment of the invention, treatment is provided for an inflammation which is intestinal, perineal or peristomal, as is for instance seen with babies or infants with diaper rash, with children or adults with diarrhoea or fecal incontinence, with patients with inflammatory bowel syndrome and with stoma patients, which all suffer from the effects of proteolytic activity which is mainly fecal.

Treatment of fecal proteolytic activity can occur by applying said inhibitor in an ointment, cream, gel, powder, or any other suitable form to the perineal or peristomal location of the inflammation. Intestinal inflammations, such as seen with IBD or pouchitis can be treated by rinsing the affected location in the digestive tract by for example administering an enema, or can be administered orally, preferably in a pharmaceutical composition, such as a draught or mixture pill, that can passage relatively unaffected through oesophagus and stomach.

These inhibitor substances can for example also be carried on wipes impregnated with an inhibitor, in sprays or in rinsing fluid. Also, it is possible to impregnate a diaper (during diaper production or shortly before use) with an inhibitor, thereby providing a method and means against diaper rash or pruritis. In a preferred embodiment, such a diaper is treated or impregnated with an inhibitor as provided by the invention in at least that diaper area (and underlying parts) that has, when in use, contact with the perineum of the baby, infant, child or adult. With diapers, said contact area normally comprises the diaper surface that is in contact with the perineum.

The invention provides a method of treatment which comprises administration to the patient or mammal prone to an inflammation of an inhibitor capable of inhibiting proteolytic activity of a protease. Inhibitors of proteolytic activity are widely known. For example, acid has an inhibiting effect on the hydrolysis of proteins by pancreatic proteases and thus a pH decreasing substance can be used as an inhibitor as provided by the invention.

Also, adsorbing substances such as activated charcoal (one such product is known as Norit), can act as protease inhibitor through their adsorbing properties. In the experimental part, several examples are given of a treatment provided by the invention whereby activated charcoal, for example Norit®, is used to treat an inflammation such as for example pouchitis.

In a preferred embodiment of the invention, the invention provides methods and means capable of inhibiting proteolytic activity of a protease. Many protease inhibitors are known (see for example G. Salvesen and H. Nagase. Proteolytic enzymes, a practical approach. Eds R. J. Beynon and J. S Bond In: The practical approach series. 1989). Although non-specific inhibitors are known (i.e. human plasma α-macroglobulin), most discriminate between protease classes or even subclasses. Substances such as peptide aldehydes or peptide chloromethyl ketones are very specific for subclasses of proteases (proteinases), depending on the peptide sequence they mimic. Others, such as metal chelators act only against metallo-proteinases or calcium dependent proteinases. Class-specific inhibitors are found against serine protease, against cysteine protease, against aspartic protease, and so on. These protease inhibitors are often commercially available as purified substances for use in biochemical preparations and may be expensive.

A preferred method according to the invention is a method wherein an inhibitor is derived from a plant, i.e. said inhibitor is a plant product comprising protease inhibiting activity. As an example, such a product derived of a plant is activated charcoal, which is obtained by burning peat or wood. A much preferred method according to the invention is a method wherein an inhibitor is derived from a plant that can give rise to fruit, seed, tubers or roots. Derived herein for example comprises derived by (partial) purification or isolation or by obtaining the necessary genetic information and producing by modern recombinant technology known in the art.

Plants often protect their leaves, fruits, seeds, tubers or roots against pests by inclusion of potent protease inhibitors and mixtures thereof in those leaves, fruits, seeds, tubers or roots. For example, cereals and legumes, such as wheat or soy beans, contain protease inhibitors such as soy bean trypsin inhibitor (SBTI), which generally has activity against trypsine or chymotrypsin but not against other proteinase classes. Tubers and roots, such as potato and cassaye, but also yam, beets and sweetroot, and others, contain potent inhibitors of a wide variety of digestive tract proteases such as aminopeptidases, carboxypeptidases, chymotrypsin, trypsin and elastase, and because of this broad range, tuber or root derived plant products comprising proteolytic activity according to the invention are preferred. Potato tubers are an extraordinarily rich source of a variety of inhibitors of all major intestinal digestive endo- and exoproteinase of animals (Pearce et al., Arch. Biochem. Biophys, 213, 456-462, 1982). Such inhibitors act as anti-nutrients that are present as part of the natural chemical defence mechanisms of plants such as tubers and roots against attacking pests. In potatoes, major inhibitors are polypeptide trypsin inhibitor (PTI), polypeptide chymotrypsine inhibitor I and II (PCI-I and PCI-II), inhibitor II against chymotrypsin and trypsine, and carboxypeptidase inhibitor, which all have analogues in other plants. These act alone and in concert against the major animal digestive proteinases.

The invention now provides a method to derive protease inhibitors from plants or plant-parts, preferably from the tubers of plants, preferably from potato tubers comprising the steps of:

a) grinding the plant or plant parts to a pulp;

b) separating the solids from the pulp to provide a juice of the plant or plant-part;

c) coagulating bulk proteins in said juice, preferably by acidifying and heating of the juice;

d) optionally binding lectins in the juice during said coagulation step by the addition of lectin-binding carbohydrates and/or oligosaccharides, preferably chitosan, to the juice;

e) separating the coagulated proteins from the juice;

f) optionally filtrating solids, e.g bacteria, from said juice and cooling the juice to ambient temperature;

f) correcting the pH to a value of the juice of about 4.0;

g) isolating the protease inhibitors from the juice by precipitating the protease inhibitors with sodium polyphosphate; and

h) optionally performing additional purification steps on the precipitate, optionally followed by neutralizing, drying and homogenizing the precipitate.

Such a method of the invention constitutes a simple process to isolate the potato protease inhibitors from potato juice water. Potato juice water is a side-stream processing material that emerges during the production of i.a. starch from potatoes, such as performed during a campaign in a potato starch factory. In the starch production process, potatoes are harvested and culled in a big storage and are transported from the storage to the factory by means of water. In a first pre-washing step sand, metals, stones, leaves etc. are removed. In a second washing step the potatoes are further washed. After washing, the potatoes are grinded by rasps with the purpose to open the cell walls. SO₂ Or sodium bisulfite, preferably an organic acid such as citric acid, more preferably ascorbic acid, is usually added to the resulting pulp, e.g. in an amount of about 100-500 ppm SO₂, or 0.1-1, e.g. about 0.2% w/v of ascorbic acid to prevent polyphenol oxidation.

Upon separation of the starch and the fibers from the pulp by using for instance centrifugal horizontal decanters (or a combination of conical centrifugal sieves and hydrocyclones) a starch/fiber cake and a centrate called potato juice water is obtained. The potato juice water may suitably be used as raw material for the isolation of crude potato protease inhibitors. In order to isolate potato protease inhibitors from the potato juice water, the potato juice water required may suitably be taken from the main transport line for potato juice water, the flow of which is usually controlled at about 300 L/h by a flow controller. The potato juice water contains about 3-6% dry matter and usually has a pH of 5.6 to 6.2.

In the process according to the present invention for the isolation of protease inhibitors from potato juice water, the bulk proteins present in the potato juice water are first coagulated, for instance by using a combination of acidification and heating of the potato juice water. Very suitably SO₂, HCl, sulfuric acid, acetic acid or citric acid, or the like, preferably citric acid, more preferably ascorbic acid is added to lower the normal pH of potato juice water from a pH of about 6 to a pH of about 3.6 to 4.4, preferably of about 4.0 to 4.2, more preferably about 4.0. The bulk proteins, mainly patatins, are then coagulated by increasing, preferably rapidly, the temperature of the acidified potato juice water to a temperature of about 50 to 70° C., preferably to about 60° C., preferably by using direct injection of steam. The total residence time for the coagulation of bulk proteins is suitably a number of seconds. Optionally, specific carbohydrates and/or oligosaccharides, preferably chitosan (poly-D-glucosamine), may be added during this coagulation step to bind lectins. The bulk of the lectins may thus also be precipitated during this step. Additionally, glycoalkaloids (TGA) may be removed prior to formation of the potato pulp by simple peeling the potatoes prior to the grinding. Lectins may alternatively be precipitated by using alcohol after the above coagulation step. A very suitable alcohol precipitation may be performed by using e.g. a final concentration of ethanol of 60 wt. %, optionally using repeated cycles precipitating and centrifugation, whereupon the pellet comprising the lectins alre removed. The alcohol may suitably be removed from the supernatant by evaporating the alcohol.

The insoluble proteins, optionally including precipitated lectins and glycoalkaloids, are subsequently separated from th rest of the potato juice water, for instance by using a centrifugal disc separator. The concentrate (insoluble fraction) of this separation step is discarded and is not used for further processing during the potato protease inhibitor isolation process.

The centrate (soluble fraction) resulting from the above separation step is optionally filtrated to remove additional solids, bacteria, etc. Any type of filter is suitable for this filtration step, including for instance a candle filter with non-reusable cartridges. The coagulation in the filtrated potato juice water is then preferably stopped, for instance by cooling the filtrate to between 1 and 30° C., preferably between 10 to 25° C., preferably by indirect cooling.

To isolate the protease inhibitors from the centrate, the protease inhibitors may be precipitated, preferably by adding to the centrate a sodium polyphosphate (NaPO₃)ₙ.Na₂O (n typically being 2 to 14), preferably n=6, and the pH is corrected to a pH of about 3.6 to 4.4, preferably about 4.0 to 4.2, more preferably about 4.0, and preferably by using the same acid as used in the first coagulation step, preferably acetic acid or citric acid, more preferably ascorbic acid. A suitable sodium polyphosphate concentration is about 1-10 wt. %, preferably around 5 wt. % and precipitation may be performed for a duration of about 60 min. A very suitable system therefore is for instance a system including a series of multiple (e.g. six) Continuous Stirred Tank Reactors (CSTR's) in cascade configuration.

The precipitated insoluble protease inhibitors are then preferably separated from the suspension by a second separation step, for instance again by using a centrifugal disc separator. The centrate of this separation step (including most of the TGA, which is still soluble at pH 4) is discarded, while the concentrate is used in the further process. By performing this second separation step, the TGA is thus effectively removed from the protease inhibitors. The concentrate, comprising the protease inhibitors is preferably washed again by diluting the concentrate with water and performing a third separation step by any suitable method, preferably by using a centrifugal horizontal decanter. The concentrate obtained from the optional second and third separation step, and comprising the insoluble protease inhibitors, may be directly used in methods or compositions of the present invention.

Alternatively, the concentrate from the third separation step is again diluted with water and neutralized towards a pH of about 7.0, including a pH range from about 4.0 to about 6.0, for instance by the addition of NaOH. More preferably, however, the pH is maintained at about 4.0, or only slightly increased to a value of about 4.8 to about 5.5.

In order to reduce salt loads of the product, the concentrate may be dialyzed against distilled water. The dialyzed concentrate may then suitably be dried in a spray dryer or any other suitable type of dryer, or may be lyophilized. The dried product may further optionally be homogenized, for instance in small batches of, e.g., about 25 kg. The homogenized product is suitably used in methods of the present invention or used to prepare compositions comprising protease inhibitors according to the present invention.

The skilled person will appreciate that analogous methods as described herein above (see for example Experimental Part II) may be used for the isolation of protease inhibitors from other plant parts, and may also be used for the isolation of protease inhibitors from fruit, seed, tubers or roots of other plants than potato. Although it is currently understood that similar protease inhibitor compositions can be obtained from other plants and/or other varieties of potato, it must be understood that the specific amount and the nature of the collection of individual protease inhibitors that would constitute such a composition will vary due to biological variation.

The invention provides the use of an inhibitor or plant product or extract capable of inhibiting proteolytic activity for preparing a pharmaceutical or personal care composition for reducing or preventing an inflammation or pruritis. In the experimental part an example is given of such a product which comprises potato juice or an inhibitor derived thereof, for example by freeze-drying. Such a composition can comprise an ointment, cream, gel, powder, or any other suitable form in which an inhibitor can be applied to a patient. In a preferred embodiment of the invention, the invention provides the use of an inhibitor or plant product capable of inhibiting proteolytic activity for preparing a composition for reducing or preventing an inflammation or pruritis which is an intestinal, perineal or peristomal inflammation or pruritis. Such a composition can be in the form of a rinsing fluid, can be contained in capsules that passage through oesophagus and stomach, can be in (prefabricated) wipes or diapers, wherein the inhibitor (or plant product) is added during production or shortly before use. In a preferred embodiment the invention provides the use of an inhibitor capable of inhibiting proteolytic activity for preparing a personal or medical care composition for perineal (perianal) and/or peristomal care, for example to counter proteolytic activity that is fecal.

For example, it is possible to prevent perineal dermatitis by rinsing the reservoir and the perineal skin with a protease inhibitor containing fluid or a protecting ointment with protease inhibitors. Also, it is possible to pre-treat diapers or personal care compositions that adsorb soiling with inhibitor powder.

The invention provides the use of an inhibitor or plant product capable of inhibiting proteolytic activity for preparing a pharmaceutical or personal care composition wherein said inhibitor or product is derived from a plant, preferably wherein said plant can give rise to fruit, seed, tubers or roots, such as a potato plant. Such an inhibitor (product, composition or mixture), as explained above, is active against a protease which is selected from the group of pancreatic and granulocyte proteases. In a particular embodiment of the invention said inhibitor (composition or mixture) is capable of inhibiting papain and/or pronase, illustrating its broad spectrum and effectivity.

The invention also provides a pharmaceutical or personal care product (for example ointments, powder, fluids) comprising inhibitors of protease activity that is capable of for example

preventing inflammation or pruritis caused by feces (fecal proteases) by inhibiting proteolytic enzymes from pancreatic and brush border origin; from bacterial (gutflora) origin; from leucocyte (granulocyte, mastcel, macrophage) origin in case of inflammation of the intestine; or

curing inflammation or pruritis caused by feces by inhibiting proteases (such as elastase, cathepsins) produced by tissue macrophages, granulocytes, mastcells; or

curing skin inflammation, and other diseases in which inflammation and disease activity is related to infiltrating inflammatory cells (effector cells) and the release of proteases; or

curing pruritis in general (treatment is often with antihistaminica) local application of ointments with protease inhibitor prevents histamine release from mastcells/protease release from fagocytes.

The invention also provides a personal care composition, rinsing fluids, wetties, powder, ointments, for peri-anal and/or peri-stomal care or a diaper comprising an inhibitor of proteolytic activity.

A pharmaceutical or personal care composition comprising inhibitors of protease activity according to the invention may be formulated specifically for topical (skin) application. The topical compositions of the present invention can be formulated in any suitable product form and may be formulated in and/or with any suitable cosmetic and pharmaceutical vehicle or carrier, including, but not limited to, aerosol spray, cream, dispersion, emulsion, foam, gel, lotion, mousse, ointment, pomade, powder, pump spray, rinsing fluid, solid, solution (both aqueous and hydro-alcoholic) and stick. One skilled in the art would generally recognize these and other standard vehicles that can be used in the present invention.

The vehicle may suitably be comprised in an article for topical administration of a skin care compositions or topical pharmaceutical compositions such as a band-aid, diaper, patch, towelette or (wet) wipe. Said article is preferably of the type having an absorbent portion wherein the topical composition comprising the inhibitor of proteolytic activity may be comprised.

The protease inhibitors are administered in a pharmaceutically effective amount. For instance they may be administered topically in unit dosage form containing about 0,1 to about 50, preferably about 0,5 to about 10 more preferably about 1 to about 5 g of protease inhibitor per day depending on the severity of the inflammation or pruritis. A pharmaceutically effective amount is defined herein as the amount of the compound required to achieve the desired bioactive effect to the patient in need of such treatment. The use of controlled release substances, for example, liposomes are especially effective.

A composition comprising inhibitors of protease activity according to the invention comprising a pharmaceutically effective amount of protease inhibitors may comprise from 1 to 20 wt. % of protease inhibitors, based on the total weight of the composition. Preferably, a composition comprising inhibitors of protease activity according to the invention comprises from about 1 to about 10 wt. %. The amount of protease inhibitors used will depend on the purity of the product obtained from the isolation procedure, with higher amounts used when the product is less pure.

A composition containing about 0.5 to 10 g of protease inhibitor in a suitable pharmaceutical vehicle is very suitable for topically treating the inflammation or pruritis.

The vehicle will typically form from 60% to 99.9%, preferably from 80% to 95% by weight of the composition, and can, in the absence of other cosmetic adjuncts or pharmaceutical adjuvants or supplements, form the balance of the composition.

A particularly useful vehicle is one that is pharmaceutically or cosmetically acceptable for topical applications. Useful vehicles include, but are not limited to, one or more water comprising aqueous systems, glycerin, C₁-C₄ alcohols, fatty alcohols, fatty ethers, fatty esters, polyols, glycols, vegetable oils, mineral oils, liposomes, laminar lipid materials, silicones, water, or any combinations thereof.

In addition, the vehicle of the compositions according to the present invention can be in the form of a homogeneous phase formulation or in the form of an emulsion including, but not limited to, oil-in-water (wherein water is the continuous phase), water-in-oil (wherein oil is the continuous phase), and multiple including triple, phase emulsions. These emulsions can cover a broad range of consistencies including thin lotions (which can also be suitable for spray or aerosol delivery), creamy lotions, light creams and heavy creams. Other suitable topical vehicles include anhydrous liquid solvents such as oil and alcohol; aqueous-based single phase liquid solvent (e.g., hydro-alcoholic solvent system); anhydrous solid (e.g. powder) and semi-solid (such as gel, cream and stick); and aqueous based gel and mousse system.

The composition may be in the form of a so-called “wash-off” product e.g. as a bath or shower gel, possibly containing a delivery system for the active principles (including the protease inhibitor) to promote adherence to the skin during rinsing. Most preferably the product is a “leave-on” product, that is a product to be applied to the skin without a deliberate rinsing step soon after its application to the skin.

Preferably the composition is a skin care solution or thin lotion that can be absorbed into a wet wipe basesheet and may include any components customary to wet wipes in order to provide desirable wiping properties.

The topical composition of the invention may optionally comprise as cosmetic adjuncts, pharmaceutical adjuvants or supplements, one or more of the following: alkalinizing agents, anesthetics, antacids, anti-allergenics, antifoaming agents, antifungals, antimicrobials, anti-inflammatory agents, antioxidants, antiperspirants, antiseptics, chelating agents, colorants, corticosteroids, depigmenting agents, emollients, emulsifiers, exfollients, film formers, fragrances (natural and artificial), humectants, insect repellents, lubricants, moisturizers, oxidizing agents, organic solvents, penetrating agents, pH buffering agents, pharmaceutical agents, photostabilizing agents, pigments, plasticizers, preservatives, propellants, reducing agents, skin protectants, skin penetration enhancers, salts, sunscreening agents, stabilizers, surfactants (or detergents), thickeners, viscosity modifiers or vitamins, or any combination thereof.

A skin care composition or topical pharmaceutical composition of the invention may optionally comprise salts to yield a solution reflecting physiological salt conditions (e.g. about 0.9% NaCl).

Compositions according to the present invention in which specific properties are desired may include as moisturizing agents, emollients, humectants, surfactants and/or emulsifiers such substances as for instance acetamide MEA, acetoglyceride, acetylated lanolin, acetylated lanolin alcohol, acrylates/C10-30 alkyl acrylate crosspolymer, acrylates copolymer, alanine, algae extract, N-alkylglycol monoisostearate, Aloe vera barbadensis Miller, Aloe vera barbadensis extract, Aloe vera barbadensis gel, Althea officinalis extract, aluminum starch octenylsuccinate, aluminum stearate, amino acids, amyl acetate, apricot (Prunus armeniaca) kernel oil, arginine, arginine aspartate, arginine pyrrolidone carboxylic acid (PCA), arnica montana extract, ascorbic acid, ascorbyl palmitate, aspartic acid, avocado (Persea gratissima) oil, barium sulphate, barrier sphingolipids, bayberry wax, beef bone fat, beef hoof fat, beef tallow, beeswax, behenic acid, behenyl alcohol, beta-sitosterol, BHT, birch (betula alba) bark extract, borage (Borago officinalis) extract, borage seed oil, 2-bromo-2-nitropropane-1,3-diol, butcherbroom (Ruscus aculeatus) extract, butyl acetate, butyl alcohol, butyl stearate, butylene glycol, cacao (Theobroma cacao) butter, calendula officinalis extract, calendula officinalis oil, candelilla (Euphorbia cerifera) wax, canola oil, caprylic/capric triglyceride, cardamon (Elettaria cardamomum) oil, carnauba (Copernicia cerifera) wax, carrageenan (Chondrus crispus), carrot (Daucus carota sativa) oil, castor (Ricinus communis) oil, castor oil fatty acid methyl ester, ceramides, ceresin, ceteareth-5, ceteareth-12, ceteareth-20, cetearyl alcohol, cetearyl octanoate, ceteth-20, ceteth-24, cetostearyl alcohol, cetyl acetate, cetyl-2-ethylhexanoate, cetyl lactate, cetyl octanoate, cetyl palmitate, chamomile (Anthemis nobilis) oil, China wood oil, chitosan PCA, cholesterol, cholesterol esters, cholesteryl hydroxystearate, chondroitin sulfate, citric acid, clary (Salvia sclarea) oil, Cocamidopropyl Betaine, coco-caprylate/caprate, coconut (Cocos nucifera) oil, collagen, collagen amino acids, copper PCA, corn glycerides, corn (Zea mays) oil, cottonseed oil, cotton wax, decyl oleate, dextrin, diazolidinyl urea, di-2-ethylhexyl sebatate, di- and triglycerides, diglycerin, di-2-heptylundecyl adipate, diisobutyl adipate, diisopropyl sebatate, diisostearyl malate, dimethicone copolyol, dimethiconol, dimethyl imidazolidinone, dioctyl adipate, dioctyl succinate, dipentaerythrityl hexacaprylate/hexacaprate, dipentaerythritol fatty acid ester, DMDM hydantoin, DNA, docosahexaenoic acid (DHA), eicosapentaenoic acid (EPA), egg oil, emulsifying wax NF, erythritol, ethoxydiglycol, ethylene glycol di-2-ethylhexylate, 2-ethylhexyl palmitate, 2-ethylhexyl succinate, ethyl acetate, ethyl laurate, ethyl linoleate, eucalyptus globulus oil, evening primrose (Oenothera biennis) oil, fatty acids, fatty acid esters, fructose, gelatin, geranium maculatum oil, germ oil, glucamine, glucosamine, glucose, glucose glutamate, glucuronic acid, glutamate, glutamic acid, glycereth-7, glycereth-12, glycereth-20, glycereth-20 stearate, glycereth-26, glycerin, glycerin di-2-heptyl undecanoate, glycerin tri-2-ethylhexylate, glyceride tri-2-heptyl undecanoate, glycerin trimyristate, glycerin trioctanate, glycerin triisopalmitate, glycerol, glyceryl behenate, glyceryl distearate, glyceryl hydroxystearate, glyceryl laurate, glyceryl linoleate, glyceryl myristate, glyceryl oleate, glyceryl stearate, glycine, glycol, glycol stearate, glycolic acid, glycosaminoglycans, grape (Vitis vinifera) seed oil, hazel (Corylus americana) nut oil, hazel (Corylus avellana) nut oil, 2-hoptylundecyl palmitate, 1,2,6-hexanetriol, 2-hexyldecyl adipate, hexyldecyl dimethyloctanate, 2-hexyldecyl myristate, 2-hexyldecyl palmitate, hexylene glycol, hexyl laurate, higher fatty acids, higher fatty acid esters, honey, hog fat, horse fat, hyaluronic acid, hybrid safflower (Carthamus tinctorius) oil, hydrocarbon oils, hydrogenated castor oil, hydrogenated coco-glycerides, hydrogenated coconut oil, hydrogenated honey, hydrogenated lanolin, hydrogenated lecithin, hydrogenated oil, hydrogenated palm glyceride, hydrogenated palm kernel oil, hydrogenated starch hydrolysate, hydrogenated soybean oil, hydrogenated tallow glyceride, hydrogenated vegetable oil, hydrolyzed collagen, hydrolyzed corn starch, hydrolyzed elastin, hydrolyzed glycosaminoglycans, hydrolyzed keratin, hydrolyzed soy protein, 12-hydroxystearic acid, hydroxylated lanolin, hydroxyproline, imidazolidinyl urea, inositol, insect wax, iodopropynyl butylcarbamate, isocetyl isostearate, isocetyl stearate, isocetyl stearoyl stearate, isodecyl oleate, isopropyl isostearate, isopropyl lanolate, isopropyl lanolin fatty acid, isopropyl myristate, isopropyl palmitate, isopropyl stearate, isostearamide DEA, isotearic acid, isostearyl lactate, isostearyl neopentanoate, Japanese wood oil, Japan wax, Japan wax nut oil, jasmine (Jasminum officinale) oil, jojoba (Simmondsia (Buxus) chinensis) oil, jojoba wax, kapok wax, kaya oil, kelp, kukui (Aleurites moluccana) nut oil, lactamide MEA, lactic acid, lactitol, lactose, laneth-16, laneth-10 acetate, lanolin, lanolin acetate, lanolin acid, lanolin alcohol, lanolin oil, lanolin wax, lauric acid, N-lauryl glutamic acid chloresteryl ester, N-lauroyl-L-glutamate-2-octyl dodecyl ester, lavender (Lavondula angustifolia) oil, lecithin, lemon (Citrus medica limonum) oil, linoleic acid, linolenic acid, linolic acid, linseed oil, liquid oil, lysine PCA, Macadamia ternifolia nut oil, magnesium stearate, magnesium sulfate, maltitol, maltose, mannitol, matricaria (Chamomilla recutita) oil, methyl gluceth-10, methyl gluceth-20, methyl glucose sesquistearate, methylsilanol PCA, microcrystalline wax, mineral oil, mink oil, monostearyl glycerin ether, montan wax, mortierella oil, myristic acid, myristyl lactate, myristyl myristate, myristyl propionate, neopentylglycol dicaproate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oil oleate, oleic acid, olive (Olea europaea) oil, orange (citrus aurantium dulcis) oil, organic acids, ozokerite, palm (Elaeis guineensis) oil, palmitic acid, pantethine, panthenol, panthenyl ethyl ether, panthenol, paraffin, PCA, PCA Glyceryl Oleate, peach (Prunus persica) kernel oil, peanut (Arachis hypogaea) oil, polyethyleneglycol (PEG)-2 stearate, PEG-2 lactamide, PEG-5 glyceryl stearate, PEG-5 soy sterol, PEG-7 hydrogenated castor oil, PEG-8 C12-18 ester, PEG-8 stearate, PEG-10 soy sterol, PEG-10 propylene glycol, PEG-15 cocamine, PEG-15 butanediol, PEG-20 methyl glucose sesquistearate, PEG-20 stearate, PEG-30 glyceryl stearate, PEG-32 stearate, PEG-40 hydrogenated castor oil, PEG-40 sorbitan peroleate, PEG-40 stearate, PEG-50 stearate, PEG-60 glyceryl isostearate, PEG-60 hydrogenated castor oil, PEG-100 stearate, PEG-150 stearate, PEG-160 distearate, pentadecalactone, pentanerythritol tetra-2-ethylhexylate, peppermint (Mentho piperita) oil, perilla oil, petrolatum, phospholipids, phytosterol, polyoxyethylene (POE) lanolin alcohol ether, POE lanolin alcohol acetate, POE cholesterol other, polyethylene glycol lanolin fatty acid, POE hydrated lanolin alcohol ether, polyamino sugar condensate, polyglyceryl-3 diisostearate, polyglyeeryl sorbitol, polyquaternium-24, polysorbate 20, polysorbate 40, polysorhate 60, polysorbate 80, polysorbate 85, potassium myristate, potassium palmitate, potassium PCA, potassium sorbate, potassium stearate, primrose oil, pristane, propylene glycol, propylene glycol citrate, propylene glycol dicaprylate/dicaprate, propylene glycol dioctanoate, propylene glycol dipelargonate, propylene glycol laurate, propylene glycol oleate, propylene glycol stearate, propylene glycol stearate SE, PVP, pyridoxine dipaimitate, quaternium-15, quaternium-18 hectorite, quaternium-22, rapeseed oil, retinol, retinyl palmitate, rico (oryza sativa) bran oil, rice bran wax, RNA, rosemary (Rosmarinus officinalis) oil, rose oil, saccharide hydrolysate, saccharide isomerate, safflower (Carthamus tinctorius) oil, sage (Salvia officinalis) oil, salicylic acid, salts of pyrollidone carboxylic acid, sandalwood (Santalum album) oil, sasanqua oil, serine, serum protein, sesame (Sesamum indicum) oil, shea butter (Butyrospermum parkii), sheep fat, shellac wax, silk powder, sodium aspartate, sodium cetearyl sulfate, sodium chondroitin sulfate, sodium DNA, sodium glucuronate, sodium hyaluronate, sodium lactate, sodium palmitate, sodium PCA, sodium polyglutamate, sodium stearate, solid oils and fats, soluble collagen, sorbic acid, sorbitan laurate, sorbitan oleate, sorbitan palmitate, sorbitan sesquioleate, sorbitan stearate, sorbitan trioleate acrylates/C10-30 alkyl acrylate crosspolymer sorbitol, soybean (Glycine soja) oil, spermaceti, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sucrose, sugarcane wax, sunflower (Helianthus annuus) seed oil, sweet almond (prunus amygdalus dulcis) oil, synthetic beeswax, TEA-lactate, TEA-PCA, teaseed oil, tocopherol, tocopheryl acetate, tocopheryl linoleate, toluic acid, trehalose, tribehenin, triglycerin, tridecyl neopentanoate, tridecyl stearate, triethanolamine, trimethylopropane tri-2-ethylhexylate, triethyl citrate, trimethylopropane triisostearate, tristearin, tsubaki oil, undecylic acid, urea, vaseline, vegetable oil, water, waxes, wheat (Triticum vulgare) germ oil, xylitol, ylang ylang (Cananga odorata) oil, etc. and mixtures thereof.

Compositions of the present invention in which specific properties brought about by surfactants are desired may include anionic surfactants, cationic surfactants, amphoteric surfactants, lyophilic nonionic surfactants and/or hydrophilic nonionic surfactants.

As anionic surfactants, for example, fatty acid soaps such as soap ingredients, sodium laurate, sodium palmitate; higher alkyl sulfate ester salts such as sodium laurosulfate, potassium laurosulfate; alkyl ether sulfate ester salts such as POE laurosulfate triethanol amine, sodium POE laurosulfate; N-acylsarcosine acids such as sodium lauroyl sarcosinate; higher fatty acid amide sulfonates such as sodium N-myristoyl-N-methyl taurine, sodium N-cocoyl-N-methyl taurid, sodium laurylmethyl taurid; phosphate ester salts such as sodium POE oleyl ether phosphate, POE stearyl ether phosphate; sulfosuccinates such as sodium di-2-ethylhexylsulfosuccinate, sodium monolauroylmonoethanol amide polyoxyethylene sulfosuccinate, sodium laurylpolypropylene glycol sulfosuccinate; alkylbenzensulfonates such as linear sodium dedecylbenzensulfonate, linear dodecylbenzensulfonate triethanol amine, linear dodecyl benzensulfate; N-acyl glutamates such as monosodium N-lauroyl glutamate, disodium N-stearoyl glutamate, monosodium N-myristoyl-L-glutamate; higher fatty acid ester sulfate ester salts such as sodium hydrogenated glyceryl cocoate sulfate; sulfated oils such as Turkey red oil; POE alkyl ether carboxylic acid, POE alkylaryl ether carboxylate, alpha.-olefinsulfates, higher fatty acid ester sulfonates, secondary alcohol sulfate ester salts, higher fatty acid alkylolamide sulfate ester salts, sodium lauroyl monoethanolamide succinate, N-palmitoyl asparaginate ditriethanol amine, sodium caseine, etc. may be used.

As cationic surfactants, for example, alkyl trimethyl ammonium salts such as stearyl trimethyl ammonium chloride, lauryl trimethyl ammonium chloride; alkyl pyridinium salts such as distearyldimethyl ammonium chloride, dialkyldimethyl ammonium chloride salts, poly(N,N′-dimethyl-3,5-methylenepiperidinium)chloride, cetylpyridinium chloride; alkyl quaternary ammonium salts, alkyl dimethylbenzyl ammonium salts, alkyl isoquinolinium salts, dialkyl morphonium salts, POE alkyl amines, alkyl amine salts, polyamine fatty acid derivatives, amyl alcohol fatty acid derivatives, benzalkonium chloride, benzethonium chloride, etc. may be used.

As amphoteric surfactants, for example, imidazoline base amphoterie surfactants such as sodium 2-undecyl-N,N,N-(hydroxyethylcarboxymethyl)-2-imidazoline, 2-cocoyl-2-imidazoliniumhydroxide-1-earboxyethyloxy-2-sodium salt; betaine base surfactants such as 2-heptadecyl-N-carboxymethyl-N-hydroxyethylimidazolinium betaine, lauryldimethyl-aminoacetate betaine, alkyl betaine, amide betaine, sulfo betaine, etc. may be used.

As lyophilic nonionic surfactants, for example, sorbitan fatty acid esters such as sorbitan monooleate, sorbitan monoisostearate, sorbitan manolaurate, sorbitan monopalmitate, sorbitan monostearate, sorbitan sesquioleate, sorbitan trioleate, diglyceryl sorbitan penta-2-ethylhexylate, diglyceryl sorbitan tetra-2-ethylhexylate; glyceryl polyglyceryl fatty acids such as glyceryl monocottonseed fatty acid, glyceryl monoerucate, glyceryl sesquioleate, glyceryl monostearate, glyceryl oleate pyroglutamate, glyceryl monostearate malate; propylene glycol fatty acid esters such as propylene glycol monostearate; hydrogenated castor oil derivatives, glyceryl alkyl ethers, polyoxyethylene methylpolysiloxane copolymers, etc. may be used.

As hydrophilic nonionic surfactants, for example, POE sorbitan fatty acid esters such as POE sorbitan monooleate, POE-sorbitan monostearate, POE-sorbitan monoolate, POE-sorbitan tetraoleate; POE sorbite fatty acid esters such as POE-sorbite monolaurate, POE-sorbite monooleate, POE-sorbite pentaoleate, POE-sorbite monostearate; POE glyceryl fatty acid esters such as POE-glyceryl monostearate, POE-glyceryl monoisostearate, POE-glyceryl triisostearate; POE fatty acid esters such as POE monooleate, POE distearate, POE monodioleate, distearate ethylene glycol; POE alkyl ethers such as POE lauryl ethers, POE oleyl ethers, POE stearyl ethers, POE behenyl ethers, POE2-octyldodecyl ethers, POE cholestanol ethers; POE alkyl phenyl ethers such as POE octyl phenyl ethers, POE nonyl phenyl ethers, POE dinonyl phenyl ethers; pluaronics such as Pluronic; polyoxyethylene-polyoxypropyleue block copolyether (POE-POP) alkyl ethers such as POE.POP cetyl ethers, POE.POP-2-decyltetradecyl ethers, POE.POP monobutyl ethers, POE-POP hydrated lanolin, POE.POP glycerin ethers; tetra-POE-tetra-POP ethylene diamine condensation products such as Tetronic; POE castor oil hydrogenated castor oil derivatives such as POE castor oil, POE hydrogenated castor oil, POE hydrogenated castor oil monoisostearate, POE hydrogenated castor oil triisostearate, POE hydrogenated castor oil monopyroglutamate monoisostearate diester, POE hydrogenated castor oil maleate; POE beeswax lanolin derivatives such as POE sorbitol beeswax; alkanolamides such as coconut oil fatty acid diethanolamide, lauric acid monoethanolamide, fatty acid isopropanolamide; POE propylene glycol fatty acid esters, POE alkylamines, POE fatty acid amides, sucrose fatty acid esters, POE nonylphenyl formaldehyde condensation products, alkylethoxydimethylamineoxide, trioleylphosphoric acid etc. may be used.

Antioxidants may for instance include compounds such as acetyl cysteine, ascorbic acid, ascorbic acid polypeptide, ascorbyl dipalmitate, ascorbyl methylsilanol pectinate, ascorbyl palmitate, ascorbyl stearate, butylhydroxyanisole (BHA), BHT, t-butyl hydroquinone, cysteine, cysteine HCl, diamylhydroquinone, di-t-butylhydroquinone, dicetyl thiodipropionate, dibutylhydroxytoluene, dioleyl tocopheryl methylsilanol, disodium ascorbyl sulfate, distearyl thiodipropionate, ditridecyl thiodipropionate, dodecyl gallate, EDTA disodium salt, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, metabisulphite, methylsilanol ascorbate, natural botanical anti-oxidants such as green tea, grape seed extracts, Bilberry extracts, and Calendula extracts, nordihydroguaiaretic acid, octyl gallate, phenyl-butyl-nitrate (PNB), phenylthioglycolic acid, potassium ascorbyl tocopheryl phosphate, potassium sulfite, propyl gallate, quinones, retinal, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

Preservatives may include e.g., butylated hydroxy anisole, methylparaben, ethylparaben, butylparaben, propyl-hydroxybenzoate; ethyl 4-hydroxybenzoate; methylhydroxybenzoate; hydroxybenzoic acid, chlorbutanol, benzyl alcohol, methylhydroxybenzoate, sodium bisulfite, sodium metabisulfite, sorbic acid, disodium EDTA, formaldehyde, phenol and the like.

Supplements may also comprise botanical extracts such as those of aloe vera, chamomile, cucumber, ginkgo biloba, ginseng, rosemary, etc.

pH adjusting agents include buffers such as for instance lactic acid-sodium lactate, citric acid-sodium citrate, succinic acid-sodium succinates, phosphate buffers, Tris buffers and the like, preferably buffers capable of buffering at a pH in the range of about 4.8 to about 5.5, more preferably about 5.0 to about 5.2.

Anti-inflammatory agents may for instance include glycyrrhizic acid derivatives, dipotassium glycyrrhizinate, glycyrrhetic acid derivatives, stearyl glycyrretinate, salicylic acid derivatives, thiotaurine, hypotaurine, hinokitiol, zinc oxide, allantoin and the like.

Anti-irritants may for instance include steroids, non-steroidal anti-inflammatories, glycyrrhizates etc.

Antacids may for instance include aluminium hydroxide, magnesium carbonate, magnesium trisilicate, magnesium hydroxide, sodium bicarbonate and calcium carbonate.

Thickeners may include for instance such compounds as agar, albumin, algae colloid (seaweed extract), alginate propylene glycol esters, aluminum magnesium silicate (bee gum), bentonite, carbomer, carboxymethyl cellulose (CMC), carboxymethyl starch, (alkyl modified) carboxyvinyl polymer (Carbopol), carrageenin, carob gum, caseine, cellulose powder, collagen, crystalline cellulose, dextran, dextrin, dialkyldimethyl ammonium sulfate cellulose, ethylcellulose, galactan, gelatin, glycyrrhinic acid; guar gum, gum arabic, hectonite, hydroxyethylcellulose, hydroxypropylcellulose, hydroxypropyl methylcellulose (Hyprome/lose), inorganic silicic acid, karaya gum, laponite, locust bean gum, methylcellulose, methylhydroxypropyl cellulose, methylhydroxpropyl starch; nitrocellulose, pectin, polyacryl amide, polyethylene glycol, polyethylene acrylate, polyethylene imine, polyoxyethylene polyoxypropylene copolymer, polyvinyl alcohol, polyvinylmethyl ether, polyvinylpyrrolidone, pullulans, PVA, PVM, PVP, quince seed (Marumero), sodium alginate, sodium cellulose sulfate, sodium pectinate, sodium polyacrylate, starch (rice, corn, potato, wheat), succinoglutan, talc, tamarind gum, tragacanth gum, xanthan gum, etc.

Antimicrobial agents may for instance include such compounds as triclosan, ethanol, doxycycline, griseofulvin, rifampicin, ampicillin, erythromycin, amoxicillin, tetracycline HCl, chloromphenicol, trimethoprim, sulfamethoxazole, sulfaphenazole, sulfisomidine, sulfadiazine, tolnaftate, sulfaguanidine, sulfadimidine, etc.

Silicones may be used as vehicle and may be included as adjunct or adjuvant for any purpose or functionality such as to improve the skin-feel, to reduce the soaping effect of the composition of the invention, as pigment dispersing aid, as solvents, as lubricant to reduce stickiness of the product and the tendency for nozzles to clog, to improving spreadability, to impart water resistance to a product or to increase its volatility. Silicones may for instance include dimethicone (dimethyl siloxane), tetramer and pentamer cyclomethicones, trimethylsilylamodimethicone, non-volatile polyalkyl siloxanes, polyether siloxane copolymers, triphenyl dimethicone, phenyl dimethicone, linear polysiloxanes such as dimethyl polysiloxane, methylphenyl polysiloxane, methylhydrogen polysiloxane, cyclic polysiloxanes such as decamethyl polysiloxane, dodecamethyl polysiloxane, tetramethyltetrahydrogen polysiloxane, silicone resins forming 3 dimensional net structures, silicone rubber, etc.

Corticosteroids may include for instance dexamethasone, betamethasone, prednisone and hydrocortisone.

As chelating agents metal ion chelates such as sodium edetate salts or EDTA may for instance be comprised in the composition.

As vitamins, vitamin A, vitamin B1, vitamin B2 (riboflavine), vitamin B6, vitamin B12 (cyano and hydroxy), vitamin C, vitamin D3, vitamin K, vitamin P, vitamin E, niacin and niacinamide, panthenols and pantothenates and folic acid may for instance be mentioned.

As penetrating agents such compounds as hyaluronic acid, insulin, liposome, or the like, as well as L-arginine or the arginine containing amino acids may for instance be used.

The cosmetic adjuncts, pharmaceutical adjuvants or supplements listed above, if present, are usually included in the compositions of the present invention at a concentration of about 0.1% to about 5.0% by weight and preferably about 1.0% to about 2.0% by weight of the composition.

The pH of the composition may be anywhere between 4.5 and 8.6. Generally for topical application the composition comprising protease inhibitors of the present invention may have a pH in the range of about 6.8 to about 7.2, i.e. a neutral pH. The pH of the composition may be adjusted or set to a value at which the protease inhibitors are most effective. Alternatively, the pH of the composition may be adjusted or set to a value at which the proteases upon which the composition acts are least active. Preferably, the pH of the topical composition of the invention has a pH in the range of about 4.8 to about 5.5, more preferably about 5.1 to about 5.3. At these values, the proteases exhibit reduced activity, and the topical composition of the invention exerts its highest effect.

To prepare the topical composition used in the method of the present invention, the usual manner for preparing skin care products or topical pharmaceuticals may be employed. The active components are generally incorporated in a dermatologically/cosmetically/pharmaceutically acceptable carrier in a conventional manner.

The components of the composition can suitably be dissolved or dispersed in an aqueous phase to be incorporated in the composition in order to prepare a lotion that can be absorbed into a wet wipe basesheet. Alternatively, to prepare a cream formulation according to the invention, the aqueous phase may subsequently be combined with an oil in the presence of a suitable emulsifier.

The composition may be packaged in any suitable manner such as in a jar, a bottle, tube, roll-ball, or the like, in the conventional manner. A further envisaged form is as a formulation suitable for delivery as a spray, either from a propellant driven aerosol or from a pump spray. Yet another envisaged form is as a formulation that is comprised in a wet towel or wet wipe, such as a formulation absorbed in a paper or cloth towel or wipe. Such wet wipes may suitably be packed in a pop-up dispenser.

Especially preferred vehicles for the topical composition of the invention may be formed by aqueous liquids, applicable in the form of wet compresses and for rinsing or cleaning purpose. Optionally, aqueous liquid compositions may comprise alcohol.

Other especially preferred vehicles include hydrogels. These semi-solid spreadable preparations contain a hydrophilic fluid such as water, glycerol, propylene glycol or alcohol, of which the viscosity is increased by inclusion of a thickener such as carbomer, hypromellose, or methylcellulose. A hydrogel has a cooling action (by evaporation of the water from the gel), is cosmetically attractive since it leaves no visible layer and is water washable. Especially the carbomer hydrogels provide easy to rub-in compositions.

Yet other especially preferred vehicles include creams. These semi-solid, spreadable preparations comprise a mixture of water and oil. Creams are emulsions of two immiscible or only partly miscible fluids wherein one (disperse phase) is comprised as very fine droplets in the other (continuous phase) by the aid of one or more emulsifiers. Creams have a emollient effect and protect the damaged skin. Examples of preferred creams are oil (disperse) in water (continuous) creams such as Lanette cream, solid Lanette cream and Cetomacrogol cream. Creams are cosmetically attractive as they do leave a barely visible layer, and they are water washable.

Other suitable, yet less preferred vehicles for the topical composition of the invention include ointments. These semi-solid spreadable preparations consist of a mixture of oils or waxes to which 25% of solids are added and may comprise an emulsifier in order to leave them water washable. Ointments have a protecting and covering action, but they do not easily penetrate into the skin as a result of which the skin feels more or less greasy and the protease inhibitors may have difficulty reaching there target area. When an ointment is to be used on or around mucous membranes a suitable additive is the thickener hypromellose, which exhibits good adherence characteristics to mucous membranes.

Other suitable, yet less preferred vehicles include alcoholic or non-alcoholic shake lotions, comprised of liquid preparations consisting of a hydrophilic fluid, usually water, sometimes mixed with alcohol or propyleneglycol, wherein a solid is finely dispersed. Shake lotions have a cooling or anti-itching action by evaporation of the water or alcohol. Upon drying they leave a fine layer of powder on the skin. Examples are Lotion Alba and Calamine Lotion.

Other suitable, yet less preferred vehicles include pastes, which are ointments with a powder or solids content of 50% or more. Pastes are ultimately suitable for wet skin applications. Preferred compositions of the invention are skin care of topical pharmaceutical compositions in the form of a hydrogel comprising 1 to 10 wt. %. of a protease inhibitor from potato; 10-20 wt. % of one or more emollients, preferably glycerol and/or propylene glycol; 1-5 wt. % of one or more thickeners, preferably carbomer, hydroxypropylcellulose, methylcellulose, hypromellose and/or tragacanth; 0.1-0.5 wt. % of one or more preservatives, preferably methylhydroxybenzoate and/or sorbic acid; and water to balance.

Other preferred compositions of the invention are skin care or topical pharmaceutical compositions in the form of an aqueous liquid comprising 1 to 10 wt. %. of a protease inhibitor from potato; 1-20 wt. % of one or more emollients, preferably glycerol or propylene glycol; 0.5-3 wt. % of one or more thickeners, preferably carbomer, hydroxypropylcellulose, methylcellulose, hypromellose and/or tragacanth; 0.1-0.5 wt. % of one or more preservatives, preferably methylhydroxybenzoate and/or sorbic acid; optionally 0.1-10% of an alcohol; and water to balance.

Yet other preferred compositions of the invention are skin care of topical pharmaceutical compositions in the form of a rinsing fluid or lotion comprising 1 to 10 wt. %. of a protease inhibitor from potato; 0.8-1.0 wt. % one or more buffers, preferably a phosphate and/or citrate buffer; 0.1-0.5 wt. % of one or more thickeners, preferably carbomer, hydroxypropylcellulose, methylcellulose, hypromellose and/or tragacanth; and water to balance.

Very good results were for instance obtained with an aqueous liquid vehicle consisting of 17 wt. % glycerol, 3 wt. % tragacanth and 0.2 wt. % methylhydroxybenzoate, balanced with distilled water.

Yet other very good results were obtained with an aqueous liquid vehicle in the form of a rinsing fluid (isotonic buffer) suitable for rinsing the peri-anal area or suitable for rinsing or patting a baby bottom. The rinsing fluid consisted of a 0.9% (w/v) phosphate citrate buffer (pH 5.2) containing 0.2% (w/v) of hydroxypropylcellulose. The vehicle was first sterilized, after which the protease inhibitors were added.

Other good results were obtained with a hydrogel vehicle consisting of 0,5-2 wt. %, 15 wt. % of propyleneglycol, 2-5 wt. % of methylpropylcellulose, 0.15% of methylhydroxybenzoate, balanced with distilled water. The pH of the hydrogel after addition of the EURO 3 highly purified protease inhibitor batch (pH 4.3) (see Experimental Part I) was around 6.0.

The method of preparation of the hydrogel may comprise a dialysis step in order to reduce the amount of salts and ascorbic acid of the protease inhibitor ingredient, which may otherwise produce a prickling gel.

Using the above vehicles, the activity of the protease inhibitors added thereto was maintained and the occurrence of dermatitis could successfully be prevented in skin test experiments.

Other good results were obtained with a hypromellose gel vehicle consisting of 60 wt. % of hypromellose, 0.15 wt. % of sorbic acid and 15 wt. % of propyleneglycol, balanced with distilled water.

Satisfactory results were obtained with a 1% carbomer hydrogel, although this vehicle became somewhat thin after addition of the potato protease inhibitors, and a Lanette cream based on 60% water, which cream further comprised vaseline, sorbic acid, sorbitol and cetiol at pH 5. The activity of the protease inhibitors was reduced by less than 50% in the Lanette cream and the preparation was capable of preventing dermatitis in skin tests.

The invention furthermore provides a protease inhibitor for use in a method according to the invention. Attention to skin care can already begin at the time of surgery, for example inhibitor containing rinse fluid. Inhibitors may be incorporated in stoma appliances, such as adhesive and absorbing discs and in stoma rinsing fluids and ointments.

Also, the invention provides a skin test for studying the effect of a protease inhibitor on proteolytic activity or inflammatory action of a substance, preferably of feces.

The invention is further described in the experimental part which is not limiting the invention.

Experimental Part I

In adults, the small intestine has a length of seven meters and the transit time of its contents is about 3 hours; this is the reason why this part of the intestine is colonised by only a few bacteria, when compared to the large intestine. However the colon is colonized by large numbers of bacteria (10¹⁰-10¹¹/gram). The transit is slow (24 hours) and the main function of the colon is absorption of water.

Finally, feces consists of one part solids and two parts of water. Half of the dry material are bacteria; the remnants are largely dietary fibre and host-derived material such as shed epithelial cells and mucus. The most active site of bacterial fermentation is the place where the contents of the ileum reaches the caecum. Abundant nutrients are available, the percentage water is high and the flora has optimal conditions to multiply. Few data about this part of the (human) intestine are known, but the pH, measured in sudden death victims, is very low (pH 4.5-5.5).

The colon flora consists for 99.9% of obligate anaerobic bacteria; anaerobic-facultative aerobic bacteria such as coliforms are a minority (about 10⁴-10⁷ bacteria/gram feces). The anaerobic colon flora is very stable and it is nearly impossible to induce alterations at species or genus level, even by drastic changes in diet (antibiotics or infection with enteropathogens however might disturb the resident flora). One of the causes of this phenomenon is that the most important nutrients derive from endogenous material, digestive fluids, mucus, etc. A part of the digestive proteins (also the bile acids) are reabsorbed from the distal part of the ileum, the remainder is converted or digested in the colon. The colonflora is thought to play an important role in the inactivation of digestive pancreatic enzymes such as proteases.

In babies and infants, the intestine is much less well developed, especially the colon does not function as well as in adults. This is the reason why digestive enzymes in feces of babies and infants are not neutralized and/or reabsorbed; its contents resemble more the contents of the small intestine, including a high proteolytic activity albeit having passed the colon.

The principal endogenous nutrient sources are probably glycoproteins from gastric and intestinal mucus which contains up to 90% carbohydrate. Bacterial glycosidases degrade the oligosaccharide side chains which protect the glycoprotein from proteolytic destruction. When the protein core lacks the protection of the carbohydrates it is no longer resistant to proteolysis by pancreatic (and bacterial) proteases. In the healthy colon there is a balance between the production and the degradation of mucus.

Much attention has been paid to inflammatory bowel diseases (IBD): Crohn's disease (CD), ulcerative colitis (UC) and pouchitis. Pouchitis is a major complication of ileoanal anastomosis with reservoir construction, after colonresection for UC and is characterized by clinical symptoms and inflammation of the reservoir (pouch). The role of the intestinal flora in IBD was investigated concerning pathogens and their contribution to degradation of the protecting mucusglycoproteins.

Patients with inflammations in the gut show a loss of the integrity of the mucosa. We have studied the potential harmful role of bacterial glycosidases and bacterial and host-derived proteases by degrading mucus glycoproteins. Therefore in patients with IBD the composition of the intestinal flora and the activity of glycosidases and proteases was estimated. Also enzymatic activity was measured in germ-free rats to establish the influence of the flora.

These studies showed that feces of patients with active CD, patients with an ileostomy and patients with a pouch have a high proteolytic activity. Proteases enter the duodenum largely as secretions from the liver, brush-border and pancreas. A part of the activity is lost in the terminal ileum, problably due to absorbtion and/or action of endogenous inhibitors. In feces of healthy subjects only a very low or no enzyme activity at all, was estimated, which is probably largely of bacterial origin. However germ-free animals such as rats show a high proteolytic activity throughout the whole large intestine. Patients with active IBD, ileostomy patients and patients with a pouch were found to have a high fecal proteolytic activity. From this we may conclude that a complete colonflora and a normal (slow) transit is necessary to inactivate these enzymes.

The high proteolytic activity in feces of patients with IBD may cause an increased degradation of mucus glycoproteins and may play a role in the maintenance of the inflammation of the mucosa. In vitro experiments confirmed this hypothesis The idea was born to treat patients such as those with an ileoanal anastomosis (IAA) with protease-inhibitors to prevent perineal dermatitis. Patients who are operated for UC or familal adenomatous polyposis (FAP), are considered for construction of an ileal reservoir after colon resection. This small reservoir is connected with the anus. The period after the operation is a hard time for most of the patients. Short after the operation the patients feces has a watery consistence, the patients are often not (yet) continent and this results in irritation and pruritis of the perineal skin (perineal dermatitis). The major cause of perineal dermatitis is the degradation of the epidermis (which consists largely of the protein keratin) by proteases.

Proteolytic activity was measured in feces of these patients and was found to be very high. Furthermore, 75% of the patients developed a moderate to severe perineal dermatitis; 25% did not have any sign of irritation.

Materials and Methods

Proteolytic Activity/Subjects

Fecal samples from twenty-seven patients with Crohn's disease (CD) were studied. Twelve patients, aged 27-58 years, had undergone intestinal surgery 3-12 years previously; locations of the resections were terminal ileum, ileum and caecum, and colon. A second group of patients was not operated; the principal sites of inflammation were ileum, ileum and colon, and colon. The diagnosis CD was established with the usual clinical, radiological and histopathological critria. All patients were outpatients.

Twelve healthy volunteers, aged 23-48 years were examined for comparison.

Ileostomy effluents were obtained from five adult patients with a conventional ileostomy (aged 38-71 years). They had undergone total colectomy more than five years before, for relief of CD or ulcerative colitis (UC), and were all currently in good health.

Fourteen patients with a pouch (median age 27 years) were studied. The patients had a restorative proctocolectomy for UC or familial adenomatous polyposis. An S pouch was constructed in 12 patients, whereas in two patients a W pouch was created. This study was performed at least one year after the restorative colectomy. The diagnosis pouchitis was based on clinical symptoms, endoscopic features of acute non-specific inflammation and histological evidence of an inflammatory cell infiltrate. Using these criteria five patients presented pouchitis and nine did not (controls).

Fecal samples from thirteen patients operated for the construction of a reservoir with ileoanal anastomosis (IAA) were collected within 14 days after the operation. Proteolytic activity was measured in feces from 31 healthy children, aged 4 months to 7 years.

Proteolytic Activity/Laboratory Animals

Feces from 4 conventional (Wistar) and 4 germ-free rats (Wag/Rij) were studied. From 2 conventional and 2 germ-free rats the contents of the intestinal tract were studied.

Fecal samples from 20 colectomized dogs, purebred Beagles (Harlan) were collected. Three ileostomy groups were studied. In ten dogs a standard Brooke ileostomy was constructed by subtotal colectomy. In five dogs a valveless ileal reservoir (pouch) was fashioned by a side-to-side iso-antiperistaltic anastomosis. After a recovery period of 2 weeks a schedule of increasing periods of occlusion was started, except for 5 dogs. The maximum tolerable occlusion time was 2.5-3 h for the ileostomy group and 4-7 h for the reservoir group. In five dogs a continent ileostomy (Kock's pouch) was constructed, which was emptied 2-5 times per 24 hours by catheterization.

Proteolytic Activity/Fecal Samples and Intestinal Contents

Feces was frozen and stored at −20° C. within 3 h of passage. Preliminary studies showed no changes in proteolytic activity during at least 4 months of storage. Samples of 1 g were transferred to 24 vol of 0.1 M phosphate buffer pH 7.6 and homogenized (‘Stomacher’, Lab blender 400). Coarse particles were removed from the homogenates by gauze filtration (Utermohlen, refolded to 2 layers); these samples are further referred to as ‘fecal homogenates’.

Immediately after killing the rats the whole intestine was removed and prepared. The small intestine was divided into 4 parts of equal length and the contents of each part was carefully washed with 0.1 M phosphate buffer (pH 7.2). Samples from coecum and colon were treated in the same way as feces.

Proteolytic Activity/Macrophages

Mouse peritoneal macrophages (RAW) were cultured in vitro in 200 ml DMEM with 5% FCS and 4 mM glutamine and stimulated with 200 U TNFαmol medium. After 18 hours the cells were harvested, centrifuged and resuspended in 2 ml 0.1 M phosphate buffer pH 7,6 Total numbers of cells were about 3.10⁸ per ml. The cells were disrupted by repeated freezing and samples were used for protease assays.

Proteolytic Activity/Enzyme Assay

Proteolytic activity was determined in the fecal homogenates in appropriate dilutions (up to 250-fold) in 0.1 M phosphate buffer (pH 7.6). Penicilline (0.1% w/v) was added to prevent bacterial growth. In the more recent inhibition tests no penicillin was used. Samples of 0.1 ml were incubated with 0.1 ml 1% (w/v) azocasein (Sigma) in phosphate buffer at 37° C. during 1 h. The reaction was stopped by addition of 0.2 ml 10% (w/v) trichloroacetic acid (TCA); after 10 min at room temperature unhydrolysed azocasein, bacteria and other particles were removed by centrifugation at 10,000 rpm during 10 min. Then 0.1 ml of the clear supernatant was transferred to 0.1 ml of 1 N NaOH in flatbottom 24 wells microplates. To the blank assays azocasein was added after incubation an addition of TCA. The absorption of the samples was measured at 450 nm and compared with standard curves obtained from solutions of azocasein. Proteolytic activity was expressed as milligrams azocasein hydrolysed during 1 h per gram dry or wet weight of sample. Each diluted sample was tested for other than enzymatic substrate hydrolysis after heating at 80° C. for 10 min. Spontaneous substrate hydrolysis was tested by incubation of the substrate with buffer.

N-succinyl-L-alanyl-L-alanyl-L-prolyl-L-leucine-p nitroanilide (Sigma) was used as substrate for estimating purified human leukocyte elastase (Sigma) and elastase activity from mouse macrophages. Samples of 0.1 ml were incubated with 0.1 ml substrate (0.1% w/v) in 0.1 M phosphate buffer pH 7.6 in a flat-well microtiter plate. After 30 or 60 min the reaction was stopped by addition of 70 μl 30% acetic acid and the absorption was measured at 400 nm. One unit of enzyme was defined as the amount which released 1 μmol of p-nitroanilide per min at 37° C.

Proteolytic Activity/Effect of pH

To test the effect of pH on the proteolytic activity the fecal samples were diluted in citric acid-phosphate buffer (0.1 M Na₂HPO₄/2H₂O, 0.1 M citric acid/H₂O) pH 5.2, 5.8, 6.8 and 7.6. Additionally the substrate solutions were made in appropriate buffers.

Preparation of Lectin-Free Potato Proteins

Crude or relatively pure potato proteins were diluted in PBS. Human erythrocytes (disease-free) were added to potato proteins (final concentration of the ery's 3%), carefully mixed for 1 min, centrifuged for 2 min at 1500 rpm. The supernatans was mixed again with the erythrocytes This was repeated 5 times until no haemagglutination was found in a haemagglutinationtest. The reciprocal value of the highest dilution of potato protein that showed definite haemagglutination was defined as the haemagglutinationtiter. The haemagglutinationtiter decreased from 25.600 to 25-1 for example. After lyophilizing, the inhibitoractivity of the lectins-free product was compared with the original protein fraction. No loss of inhibitor activity was found when tested in fecal samples with a high proteolytic activity and in purified protease solutions (trypsin, α-chymotrypsin and elastase, final concentration 1%).

Furthermore lectins-free potato proteins are obtained by using for example chitooligo-agarose (Seikagaku).

Lectins from potato proteins may also be removed by applying alcohol precipitation (e.g. 60% ethanol) procedures as described above.

Lectins from potato proteins are also inactivated, not by removing them from the protein solution, but by binding to soluble carbohydrate moieties, such as for example N-acetochitooligosaccarides from hydrolized chitin and glycopoteins from stomach or intestine. The lectines are still in the product but have lost their active site.

Proteolytic Activity/Protease Inhibitors

The following inhibitors were used:

Trasylol (Aprotinin) (Bayer) not diluted

Ovomucoid ( )1% (w/v) in 0.1 M phosphate buffer pH 7.6

Foetal Calf Serum (FCS) ( ) not diluted

Trypsin inhibitor II-from Soybean (STI) (T-9003; Sigma) 1% (w/v) in phosphate buffer pH 7.6

Norit A (supra USP, 951191), B (Test BUR, A6910), E (Supra USP, 940260), PRSH, Carbomix, tablets

Premium powder (Hollister)

Alternatively, potato juice (PJ) from “Bintjes” was prepared as follows. After peeling and washing, the potatoes were smashed to pieces, filtered through cambric under addition of 0.2% ascorbic acid. The juice was centrifuged at 27,500 RCF for 30 min at 4° C., filtered through paper and again centrifuged. The clear yellow supernatans was filtered through a 0.45 micron filter and freeze-dried. This crude product was sterile (controlled with bloodagarplates) and contained about 25% protein. Ten gram PJ powder was derived of 200 ml juice.

Potato juice (PJ) from “Bintjes” was prepared as follows. After peeling and washing, the potatoes were smashed to pieces, filtered through cambric under addition of 0.2% ascorbic acid. The juice was centrifuged at 27.500 RCF for 30 min at 4° C., filtered through paper and again centrifuged. The clear yellow supernatants was filtered through a 0.45 micron filter and freeze-dried. This crude product was sterile (controlled with blood agar plates) and contained about 25% protein. Ten gram PJ powder was derived of 200 ml juice.

In general, protease inhibitors which are present in potatoes for example can be recovered by grinding potatoes, removing starch and other solids, and for example freeze-drying the juice.

The purity of the protease inhibitor preparation can be improved by removing non-proteinaceous material and/or low molecular weight peptides and/or amino acids present in potato juice by e.g. centrifugation, microfiltration, ultrafiltration, diafiltration or electrodialysis. Furthermore, protein can be selectively recovered in a relatively crude form from the potato juice matrix. This can be achieved by e.g. ultrafiltration, iso-electric precipitation, (co)flocculation with polyelectrolytes or any other flocculation aid, coprecipitation with other proteins, protein precipitation with salt (salting out), or by changing the quality of the solvent e.g. by adding aceton, methanol, ethanol or iso-propyl-alcohol, by iso-electric precipitation and thermal fractionation and other techniques known to anyone skilled in the art. Since protease inhibitors in potato juice are relatively heat stable, a moderate thermal treatment leads to denaturation and coagulation of less stable proteins. Coagulated protein can subsequently be removed by techniques as simple as e.g. centrifugation. Although some protease-inhibiting activity is lost, the purity of the remaining protease inhibitors is increased. Even further purification is possible by ultrafiltration or by salting out the protease inhibitors, and subsequent removal of salt and other undesired components by ultra- and diafiltration. Alternatively, isolation of several protease inhibitors is possible by affinity chromatography, either directly from the crude potato juice matrix or after pre-purification.

In most of the experiments the inhibitor was added to feces (diluted 1:25 in buffer), mixed for 5-15 min and added to the substrate; PJ-powder was added to undiluted feces (1:1) and after mixing, diluted (1:25) with buffer. In each experiment controls were assayed (sterilized feces, sterilized inhibitors, buffer solutions).

Proteolytic Activity/Purified Enzymes

The following enzymes were tested in the inhibition experiments:

bovine pancreatic trypsin (Serva)

bovine pancreatic α-chymotrypsin (Merck, Sigma)

bovine pancreatic elastase (Sigma)

papaine (Sigma)

pronase (Sigma)

(carboxypeptidase and leucinaminopeptidase were tested, but did not hydrolyze azocasen)

All enzymes were used in a concentration of 0.2% (w/v) in buffer.

Proteolytic Activty/Skin Tests

Skin tests were performed on the ventral part of the fore-arm. The following solutions were tested: 1. supernatant from feces of a patient with an ileum reservoir with a high proteolytic activity; 2. the same supernatant, but sterilised; 3. supernatant with 0.25% STI (w/v) and 4. 0.25% STI in buffer. Two hundred μl of each solution were placed on folded cambric on the skin and covered with plastic and adhesion wound pad. Total incubation time was 7 h, but after 3 and 5 h 100 μl buffer was added to each of the test patches to prevent dehydration.

Inhibition of Fecal Proteolytic Activity by Products from Potato Juice Euro 1, Euro 2, Euro 3

Euro 1 is crude PJ powder, Euro 2 and 3 are more purified.

Fecal Samples

Feces from 1 patient with an ileostomy, 1 patient with a good-functioning pouch, 1 patient 14 days after colectomy and the construction of a pouch, 2 babies aged 4 months were used.

Feces were used undiluted except for the babies, which was diluted 1:1 in phosphate buffer pH 7.6 and centrifuged 10 min at 10,000 g.

EURO's

EURO's were used as 1:5, 1:10, 1:25, 1:50 and 1:100 dilutions in phosphate buffer pH 7.6.

Feces and EURO were mixed 1:1 for 10 minutes, then the mixture was diluted in phosphate buffer pH 7.6 1:12.5.

In both dilutions proteolytic activity was measured with azocaseine as substrate.

Skin Tests

Patch Test Chambers (van der Bend) of 10 by 10 mm, filed with 50 μl of a test solution were placed on the skin of the upper part of the back of 2 healthy subjects and fixed with Fixomull Stretch self adhesive tape; the distance between them was 15 mm. One series of 4 test chambers was placed from cranial to caudal, a second series from caudal to cranial. The test solutions had the following composition:

A. elastase, trypsin and α-chymotrypsin, end concentration of each of the enzymes 1% (Enzyme Mix) soluted in sterilized fecal supernatant from an ileostomy patient (PS)

B. FS

C. Euro 2 (end concentration 5%) dissolved in FS with Enzyme Mix

D. Euro 2 in FS

After 24 hours the test chambers were removed and the skin was rinsed with tap water. Sites were inspected for erythema and dermatitis after 1, 2, 4, 6 and 24 hours.

A comparable skin test was made with the more purified potato protein fraction (EURO 3), end concentration 1%. A fifth test chamber was placed to control contact dermatitis: E (potato protein in distilled water). Twelve healthy subjects were tested.

Allergy Tests

Type 4 (contract dermatitis): 31 patients of the department of Dermatology (AZR) were tested with the relatively purified (Euro 3) potato protein according standard protocols.

Type 1 (IgE mediated): prick tests: 10 patients of the department of Allergy (AZR) with food allergy were tested and 1 patient with a severe allergy towards potato protein.

Results

1 Proteolytic Activity in Feces

Proteolytic activity in feces from healthy subjects was low. However Table 1 shows that patients with CD, ileostomy patients and patients with a pouch (with and without pouchitis) have a high proteolytic activity.

TABLE 1
Proteolytic activity in feces of healthy subjects and patients

| | | | Dry weight of feces | |
|---|---|---|---|---|
| | Proteolytic activity | | mg/g | |
| | median | (range) | median | (range) |
| Healthy subjects | 17.9* | (7.5-44.0) | 313 | (164-403) |
| Patients with CD | | | | |
| no resections | 47.9 | (19.1-192.0) | 216 | (128-228) |
| resections | 228.7 | (130.6-356.6) | 134 | (84-175) |
| Patients with | 336 | (89-972) | 88 | (69-120) |
| ileostomy | | | | |
| Patients with a pouch | | | | |
| no pouchitis | 14** | (5.5-23.5) | 83 | (57-103) |
| pouchitis | 14 | (7.1-17.3) | 52 | 30-110) |
| Patients with | 53 | (18-105) | ND | (<30) |
| IAA | | | | |

*azocasein hydrolyzed, mg/h/g dry feces
**azocasein hydrolyzed, mg/h/g wet feces

Comparable results were found in fecal samples of laboratory animals. Feces from normal dogs and rats had a very low proteolytic activity. Proteolytic activity was found to be high in ileostomy output and in valveless pouches of dogs despite occlusion; however continent pouches showed a complete normalization concerning the proteolytic activity (and several other parameters which are not discussed in this context). In contrast with germ-free rats in the colon of conventional animals, the proteolytic activity is strongly decreased, which suggests a role for the colon flora in inactivation (and/or degradation) of digestive proteases. In infants, proteolytic activity varies with age. An estimate of the proteolytic activity in feces of healthy infants and children show in infants (n=10, 4-12 months) very high activity, in children (n=9, 1-2 years) lower, but still high activity and in children (n=12, 2-8 years) decreasing activity.

In a further experiment, the proteolytic activity in feces of 31 children was again found to decrease with age, in children of 4 months (n=4): 191 mg hydrolyzed azocasein/h/g feces, in children of 6 months (n=2):109 mg, in a child of 8 months (n=1): 118 mg, in children of 11 months (n=8):105 mg, in children of 16 months (n=3): 78 mg, in children of 24 months (n=6): 34 mg, in children of 3 years (n=5): 24 mg, in children of 5 years (n=3): 3 mg, in children of 7 years (n=4): 14 mg was found.

2 Inhibition of Proteolytic Activity

pH

FIG. 3 shows that the pH dependence of the proteolytic activity was similar in each of the tested samples. At pH 6.8 and 7.6 the activities were respectively three and four times higher than at pH 5.2 (p<0.001 for both comparisons). This means that at pH of 5.2 the proteolytic activity is inhibited for 75%.

STI

The next table (Table 2) shows the results of our first experiments with protease inhibitors. Conditions of the assays were different but Trasylol, ovomucoid and FCS had effects on the proteolytic activity which were less promising or (conflicting) than STI. In a concentration of 1% (w/v) the inhibition was more to 80%.

TABLE 2
Inhibition of proteolytic activity in patients and dogs

| | % inhibition of the proteolytic activity | | | | | |
|---|---|---|---|---|---|---|
| | CD patients* | | Ileostomy dog | | pouch dog | |
| | n = 4 | n = 1* | n = 3° | n = 1† | n = 2⁺ | n = 1** |
| ovomucoid | 68 | 93 | 84 | 52 | | |
| trasylol | | | 54 | 56 | | 16 |
| STI 1% | | 93 | 84 | | | |
| (0.25, 0.5, | | | (63, 61, 45) | | | |
| 0.75%) | | | | | | |
| FCS | | 94 | 0 | | | |
| ovomucoid + | | | 51 | | | |
| trasylol | | | | | | |
| ovo + tras + | | | | 76 | | |
| STI | | | | | | |
| ovo + STI | | | 70 | | | |
| norit A | | | | 50 | | |

*inhibitor added to feces 1:2000 diluted; 20 h incubated with substrate
°inhibitor added to feces diluted 1:100; 2 h incubated
†undiluted feces + inhibitor (3 + 1), mixing for 2 h, then diluted 1:100
⁺undiluted feces + norit (4 + 1) mixing for 2 h (or 13 min), then diluted 1:100
**2 g feces + 0.5 ml trasylol; mixing for 15 min, then diluted

Norit

Several kinds of norit were tested with feces from pouch patients with a high proteolytic activity for optimal adsorbing qualities, to be used as protease inhibitor in fluid to rinse IAA patients after their operation. In this experiment Premium powder was tested also. Table 3 shows that the adsorbing capacities of norit PRSH and norit E for proteases were extremely strong.

TABLE 3
Effect of norit on proteolytic activity in feces

| | % inhibition of the proteolytic activity* |
|---|---|
| Carbomix | 0 |
| Norit A (Serva) | 83 |
| Norit A | 83 |
| Norit B | 0 |
| Norit E | 97 |
| norit PRSH | 100 |
| norit tablets | 58 |
| premium powder 17 | |

*feces from 7 patients was diluted 1:25 with buffer with 5% norit; before addition of substrate the mixture was centrifuged (norit also may adsorb the substrate); values are medians.

Inhibition of the proteolytic activity by norit was confirmed by using skimmed milk plates; the caseine in the agar is hydrolyzed by proteases and clarification is seen after treatment with TCA.

Norit PRSH was tested in different concentrations at pH 5.2 and 7.6.

TABLE 4
Effect of Norit PRSH on proteolytic activity in feces

% Inhibition of the proteolytic activity

| | pH 5.2 | ph 7.6 |
|---|---|---|
| Norit PRSH 1% | 76 | 36 |
| 2% | 95 | 92 |
| 3-5% | 100 | 100 |

Potato Juice (PJ)

PJ was initially prepared and tested as fluid; later on a freeze-dried product was prepared. The initial end concentration of the PJ powder in the fecal suspensions was 17%. Table 5 shows the inhibition of fecal proteolytic activity by PJ and PJ powder.

TABLE 5
Effect of PJ on proteolytic activity of feces

| | % Inhibition of the proteolytic activity | | | |
|---|---|---|---|---|
| Number of patients | 4* | 4° | 7† | 7⁺ |
| PJ | | | | |
| undiluted | 93 | (50) | | |
| 1:5 diluted | 90 | | | |
| 1:10 diluted | 51 | | | |
| 1:25 diluted | 23 | | | |
| PJ powder | | | | |
| 17%(w/v) | | | 88 | 97 |
| 10% | | | 78 | 90 |
| 5% | | | 53 | 74 |
| 2% | | | 20 | 44 |

*feces 1:12.5 diluted in buffer, then mixed with Pj(1 + 1)
°feces and PJ 1:1 mixed for 15 min, centrifuged, 1:25 diluted
†feces and PJ powder (1 g in 2 ml buffer) 1:1; this gives an end concentration of 17%; no further dilutions for the assay
⁺same experiment as †, but the mixture was diluted 1:25 for the assay

Heating of the PJ powder in a solution of 1 g in 4 ml buffer (end concentration in feces 5%) did decrease the inhibitor capacities as follows:

| unheated PJ: | 65% inhibition of the proteolytic activity |
|---|---|
| 30 min at 55° C.: | 64% |
| 30 min at 80° C.: | 47% |
| 30 min at 90° C.: | 24% |
| 30 min at 100° C.: | 14% |

Effect of protease inhibitors on the activity of pure enzymes is shown in Table 6. PJ powder is a very potent inhibitor of several pancreatic enzymes, papain and pronase.

TABLE 6
Effect of protease inhibitors on purified enzymes

| | % Inhibition of the proteolytic activity | | | | |
|---|---|---|---|---|---|
| | trypsin | chymotrypsin | elastase | pronase | papain |
| STI (0.125%) | 99 | 99 | 15 | | |
| STI-A | 100 | 80 | 50 | 0 | 0 |
| STI-B | 100 | 100 | 60 | 0 | 0 |
| STI-C | 100 | 100 | 55 | 0 | 0 |
| Trasylol | 95 | 95 | 10 | | |
| (undil.) | | | | | |
| PJ* | 100 | 100 | 100 | 38 | 83 |
| PJ | | | | | |
| (30 min at | | | | | |
| 80° C.) | | | | | |
| 1:5 | | | 100 | | |
| 1:10 | 100 | 100 | 97 | | |
| 1:30 | | | 95 | | |
| 1:40 | | | 94 | | |
| 1:50 | | | 91 | | |
| 1:75 | | | 90 | | |
| 1:100 | | | 88 | | |
| 1:1000 | | | 54 | | |

*1 g PJ powder was mixed with 2 ml and with 4 ml buffer

Testing of Trypsin Inhibitors from Soybeans

STI-A: STI-type I-S Sigma T 9003

STI-B: STI-type II-S Sigman T 9128

STI-C; Bowman-Birke Inhibitor Sigma T 9777

enzyme concentration was 0.02%, inhibitor concentration (end concentration) was 0.125%.

Possible interactions of the PJ-inhibitor with the substrate was tested by using different concentrations of azocasein in the same experiment. No interactions were found:

TABLE 7

% Inhibition of elastase (0.02%)-activity

| PJ diluted: | 1% azocasein | 2% azocasein |
|---|---|---|
| 1:50 | 100 | 100 |
| 1:100 | 97 | 95 |
| 1:500 | 87 | 75 |
| 1:1000 | 65 | 66 |
| 1:2000 | 44 | 51 |

Skin Tests

After removing the pads and the cambric the skin was carefully cleaned with tap water and judged immediately and after 1-18 h. No reaction was seen with sterilized feces (2) nor with STI in buffer (4), however moderate redness, papulas and some vesiculas could be observed at location 1 (fecal supernatant). Location 3 (fecal supernatant with STI) showed a slight redness that disappeared within 60 min.

The effect of potato juice or inhibitors derived thereof was also tested in vitro and in a skin test. The results are shown in FIGS. 1-6. It is possible to inactivate 90-100% of total proteolytic activity, extracts from potato, such as potato juice or inhibitors derived thereof are able to inactivate fecal proteases and this prevents inflammation.

In the skin test, PJ, and its various purified fractions were shown to be very effective when applied to treat and prevent an inflammation. Whereas as sterilized fecal supernatant from an ileostomy patient caused an inflammation of the skin, and a severe dermatitis (redness, oedema, vesiculas, pain) when proteolytic enzymes were added, no inflammation was found when potato juice inhibitor was added in both cases. For example ointments, creams or gels, when mixed with PJ inhibitor, are capable to inhibit or prevent the local dermatitis.

Furthermore, no allergic or other adverse reactions where observed against both the potato juice product.

Inhibition of Macrophage Proteases

The production of proteolytic enzymes by mouse macrophages was stimulated by TNFα. Addition of purified potato proteins (EURO 3) inhibited the activity of elastase-like proteases for 70%

Elastase Activity in mU

| | |
|---|---|
| Macrophages (6.10⁸ cells) | 26.5 |
| Macrophages (6.10⁸ cells) + EURO 3 (1%) | 8.0 |

These results show that the activity of (purified) human leucocyte elastase is reduced by potato protease inhibitors

Discussion

Furthermore these experiments show that patients with intestinal inflammations and/or resections of the colon or ileum, but also infants and children up to 2 years of age have a high fecal proteolytic activity. These enzymes are of pancreatic, brush-border, microbial and/or cellular (granulocytes, macrophages) origin. These enzymes impair the protective intestinal mucuslayer as well as the skin in the perineal zone.

Both crude and purified potato proteins (protease inhibitors) inhibit the activity of fecal proteases (hydrolyzing azocasein) and the activity of macrophage elastase (hydrolyzing N-succinyl-L-alanyl-L-alanyl-L-prolyl-L-leucin-p nitroanilide. Purified pancreatic enzymes, trypsin, chymotrypsin and elastase are also inhibited by PJ(crude or purified)

In a skintest dermatitis developed within 24 h using purified pancreatic proteases dissolved in sterilized fecal supernatans. This test is microbiologically safe, but the additional effects of fecal compounds, such as bile acids are intact. Dermatititis was completely prevented by the addition of crude or purified potato protease inhibitors to the testsolution.

It is probably not wise to use fecal supernatans again (for reasons of safety), but a mixture of the 3 purified enzymes in appropriate concentrations has the same effect. Azocasein is a substrate which is hydrolysed by several hydrolytic enzymes, but also enzyme specific substrates can be tested. STI is just one of the inhibitors from soybeans, inhibiting trypsin and chymotrypsin, but not elastase.

Experimental Part II

In order to degrade dietary proteins, the stomach, the pancreas and the small intestinal brush-border secrete several classes of proteases. These enzymes are transported through the small intestine in about three hours. A part of their activity is lost in the distal ileum, probably due to absorption and the action of endogenous inhibitors. However, a still considerable amount of active proteases enters the large intestine. During their stay in the colon (about 24 hours), the activity of the enzymes is greatly reduced, probably by the action of a special consortium of colon bacteria. Neutralized and digested remnants of food, endogenous waste and bacteria finally leave the body via the rectum. In feces from healthy subjects a very low proteolytic activity, probably of both bacterial and pancreatic origin, was measured.

Only when the colon cannot effectively reduce the intestinal water content and neutralize the enzymes, the feces may still contain a high proteolytic activity, which may cause irritations to the intra-anal and peri-anal skin during periods of diarrhea or fecal incontinence.

The human skin, protected by the stratum corneum, which consists of protein enriched corneocytes embedded in an intracellular lipid matrix, is probably very susceptible to degradation by fecal components, such as non-inactivated proteases; also bile acids, small amounts of pancreatic lipase and bacterial antigens, may influence this process. We hypothesize that pancreatic proteases from feces are the major cause of peri-anal dermatitis in patients with diarrhea. More or less liquid stools are a temporary problem during gastrointestinal infections, but are often very damaging for patients who have undergone resections of colon and/or ileum. In the elderly, fecal incontinence is a major problem with serious consequences. A study among nursing home residents revealed that fecal incontinence was a major risk factor associated with the formation of stage II-IV pressure ulcers.

Treatment of feces-induced inflammations is mainly based on providing an either protective layer to the skin, e.g. by applying a lipid-based ointment, containing additives such as zinc or aluminum, or by general anti-inflammatory therapy which often resorts to the application of corticosteroids, despite the serious side-effects that are often seen. However, none of these treatments does more than alleviate the clinical symptoms.

The aim of the present experiment was to assess the effectiveness of the protease inhibitors in treating and preventing peri-anal dermatitis by inhibiting the fecal proteases.

In preliminary experiments we tested several potential non-toxic protease inhibitors for their ability to inhibit the main intestinal pancreatic proteases, trypsin, α-chymotrypsin and elastase (Sigma, Chemical Company, MO). Except for potato juice, which inhibited the activity of these three enzymes nearly completely, the other inhibitors, soy bean trypsin inhibitors (type I-S, type II-S, Bowman-Birke; Sigma), aprotinin (Trasylol; Bayer, München, Germany), ovomucoid (Sigma) and fetal bovine serum (Sigma) suppressed the larger part of the trypsin- and α-chymotrypsin activity, but only 10-55% of the activity of elastase. Therefore potatoes were chosen to prepare a product containing protease inhibitors, in order to suppress the fecal proteolytic activity.

Materials and Methods

Subjects

Feces samples from twenty healthy volunteers, aged 23-48 years, and from thirty-one healthy children aged 4 months to 8 years were studied. Ileostomy effluents were obtained from 8 ileostomists with a conventional ileostomy (aged 38-71 years). They had undergone total colectomy more than 5 years before, for relief of Crohn's disease (CD) or ulcerative colitis (UC), and were currently in good health. None of the subjects was on a restricted diet or receiving medication.

Fourteen patients with an ileum reservoir (pouch), aged 22-45 years were studied. The patients had a restorative proctocolectomy for UC or familial adenomatous polyposis (FAP). An S pouch was constructed in twelve patients, whereas in two patients a W pouch was created. This study was performed at least one year after the operation. The patients were currently in good health and were free of medication except for one patient who required treatment with mesalasine (Pentasa®, Yamanouchi, Leiderdorp, The Netherlands).

Feces from fourteen patients with CD, aged 27-58 years, was examined. The diagnosis CD was established with the usual clinical, radiological and histopathological criteria. The principal sites of inflammation were ileum, ileum and colon, and colon. The patients had undergone intestinal surgery 2-12 years before. All patients were outpatients and currently in good health. Five patients were treated with mesalasine; the other patients did not receive medication for the last 3 months.

From each subject of the above mentioned groups, two random samples of feces or ileostomy effluent were obtained with at least a 2-month interval.

Four to six fecal samples from each of twenty hospitalized patients, successfully operated for UC or FAP, aged 25-45 years, were collected between 1 and 15 days after the construction of a reservoir (pouch) with ileoanal anastomosis (IAA). The development of peri-anal dermatitis of 48 IAA patients was studied by daily inspection during their stay in the hospital.

Ten healthy volunteers, without a history of (atopic) dermatitis, aged 27-56 years, were included in the skin tests. Informed consent of the subjects was required and the study was approved by the Ethical Committee of the Academic Medical Center of the Erasmus University of Rotterdam (Netherlands).

Proteolytic Activity in Fecal Samples

Fecal samples for estimation of proteolytic activity were prepared as follows. Immediately after defecation (or after removal of the ileostomy bags), the feces was transported to the laboratory and stored at −20° C. Preliminary studies showed no changes in proteolytic activity during at least four months of storage. Samples of 1 g were transferred to 24 ml of 0.1 M phosphate buffer pH 7.6 and homogenized (“Stomacher”, Lab blender 400, Seward, Bury St. Edmunds, England). Further dilutions were made in phosphate buffer also. Coarse particles were removed from the homogenates by cambric gauze filtration (refolded to 2 layers).

Proteolytic activity was determined in the fecal homogenates in appropriate dilutions in 0.1 M phosphate buffer (pH 7.6). Samples of 0.1 ml were incubated with 0.1 ml 1% (w/v) azocasein (Sigma) in phosphate buffer (pH 7.6) at 37° C. for 1 h; it was established that the reaction rate was linear. The reaction was stopped by addition of 0.2 ml 10% (w/v) trichloroacetic acid (TCA; Sigma); after 10 min at room temperature unhydrolyzed azocasein, bacteria and other particles were removed by centrifugation at 10,000 rpm during 10 min. Then 0.1 ml of the clear supernatant was transferred to 0.1 ml of 1 N NaOH in flatbottom 24 wells microplates. To the blank assays azocasein was added after incubation and addition of TCA. The absorption of the samples was measured at 450 nm and compared with standard curves obtained from a titration series of azocasein. Proteolytic activity was expressed as milligrams azocasein hydrolyzed during 1 h per gram feces. The limit of detection was 0.125 mg hydrolyzed azocasein/h. Each diluted sample was tested for other than enzymatic substrate hydrolysis after heating at 80° C. for 10 min. Spontaneous substrate hydrolysis was tested by incubation of the substrate with the buffer.

Elastase Activity from Macrophages

Samples for estimation of proteolytic enzymes produced by macrophages were prepared as follows. Mouse peritoneal macrophages (RAW) were cultured in vitro in 200 ml Dulbecco's modified Eagle's medium (DMEM; Bio-Whittaker Europe, Verviers, Belgium) supplemented with 5% fetal bovine serum (Sigma) and 4 mM glutamin (Sigma) and stimulated with 200 U TNF-α (Santa Cruz Biotechnology, Santa Cruz, Calif.)/ml medium. After 18 h of incubation at 37° C. the cells were harvested, centrifuged at 1500 rpm for 5 min and resuspended in 2 ml 0.1 M phosphate buffer pH 7.6. Total numbers of cells were about 3.10⁸ per ml. The cells were disrupted by repeated freezing at −20° C. and slow thawing on ice.

N-succinyl-L-alanyl-L-alanyl-L-prolyl-L-leucine-p-nitroanilide (SAAPLPNA; Sigma) was used as the substrate for estimating elastase activity from mouse macrophages and purified human leucocyte elastase (source: human leukocytes; specific activity: ≧50 units/mg protein; E 8140, Sigma).

Samples of 0,1 ml were incubated with 0.1 ml substrate (0.1%, w/v) in 0.1 M phosphate buffer pH 7.6 in a flat-well microtiter plate at 37° C. After 30 or 60 min the reaction was stopped by addition of 70 μl 30% acetic acid. The samples were centrifuged at 10,000 rpm for 10 min and the absorption was measured at 405 nm. One unit of enzyme activity was defied as 1 μmol of released p-nitroanilide per min at 37° C. Elastase activity was expressed as U per ml, corresponding to a lysate of 3.10⁸ cells. The limit of detection was 0.2 μmol of p-nitroanilide per min.

Preparation of Protease Inhibitor Fractions from Potatoes

From potatoes (Solanum tuberosum) variety “Bintje” a crude (fraction 1) and a more purified fraction (fraction 2) were prepared.

After peeling and washing the potatoes were cut to pieces and homogenized in a braunshaker (Braun A G, Frankfurt/M, Germany), filtered through cambric gauze and 0.2% (w/v) ascorbic acid (Sigma) was added to the filtrate. The juice was centrifuged at 13.000 rpm for 30 min at 4° C. The supernatant was heated for 15 min at 65° C. in a waterbath, to inactivate enzymes such as phenoloxidase which cause brown coloring, and after cooling, centrifuged and filtered through paper. This filtrate was freeze-dried and is further referred as fraction 1. This crude product contained about 25% protein. The BCA-200 Protein Assay Kit (Pierce, Rockford, Ill.), with bovine serum albumin as a standard, was used to determine proteins in the potato fractions.

Fraction 2 was prepared by addition of ammonium sulfate to 55% saturation at 4° C. to the above described filtrate (fraction 1 before freeze drying). The precipitate was allowed to settle overnight and then collected by centrifugation for 15 min at 10,000 rpm at 4° C. The precipitate was dissolved in water and dialysed extensively against water. The resulting precipitate was removed by centrifugation for 10 min at 10,000 rpm at 4° C. The supernatant was lyophilized. This fraction consisted of 60% protein.

Fraction 1 and 2 were controlled for microbiological contamination by seeding 0.1 ml of a 20% solution (w/v) on blood agar- and malt agar plates (Oxoid, Basingstoke, England). After 48 hours of incubation at 20 and 37° C. the plates were read. Those factions that did not show growth were used in the experiments.

Inhibition of Proteolytic Activity in Feces by Potato Protein Fractions

Fecal samples from patients with an ileostomy and a pouch, and feces from patients 10 days after IAA were centrifuged at 13,000 rpm for 10 min at 4° C. Feces from 4 months old babies were diluted 1:1 with phosphate buffer pH 7.6 before centrifugation.

Potato-protein fraction 1 and fraction 2 were diluted in phosphate buffer pH 7.6 to a concentration of 200, 100, 40, 20 and 10 mg/ml.

The fecal supernatant and the dilutions of fraction 1 and 2 were mixed 1:1. After 10 min of incubation on a rocking plateau at room temperature, phosphate buffer was added to a part of the mixtures resulting in a 1:12.5 dilution. In both mixtures and in controls without potato proteins, the proteolytic activity was measured with azocasein as the substrate. Proteolytic activity was expressed as mg hydrolyzed asocasein per hour per gram (undiluted) feces.

Characterization of Protease Inhibitor Fraction 2

Gel Filtration

Three gram of fraction 2 was dissolved in 20 ml distilled water and centrifuged at 10,000 rpm for 20 min. The clear supernatant was applied to a Superdex 75 column (XK50/100, 1700 ml) using an Akta Purifier chromatography system (Pharmacia Biotech, Sweden). The gel was equilibrated with 2 litre 25 mM Tris-HCl buffer pH 7.0 and proteins were eluated with the same buffer. Fractionation was performed with a flow of 2 ml per min. The absorbance of the eluates was determined at 280 nm. From 400 ml (void volume) fractions of 18 ml were collected, resulting in 126 fractions. All fractions were sterilized using low protein binding 0.22 μm filters (Millipore S. A., Molsheim, France) and controlled for microbiological contamination as described above. No growth on agar media was observed. Each of 126 fractions was tested for the presence of inhibitors of pancreatic trypsin, α-chymotrypsin and elastase.

Protease Inhibition Assays

To determine the inhibitor activity, 0.1 ml fraction (undiluted, 10 and 100 times diluted) was mixed with 0.1 ml enzyme solution and incubated for 10 min at room temperature. The enzyme solution consisted of 10 μg/ml trypsin (source: porcine pancreas; specific activity: 15.9 units/mg protein; T0134, Sigma), 1 μg/ml achymotrypsin (source: bovine pancreas; specific activity: 40-60 units/mg protein; C 7762, Sigma) or 7.5 pg/ml elastase, (source: porcine pancreas; specific activity: min. 1 units/mg protein; E 68883, Sigma). After addition of 0.2 ml 2.5 mM p-nitroanilide substrate (respectively N-α-benzoyl-L-arginine-pNa, N-succinyl-L-alanyl-L-alanyl-L-prolyl-L-phenylalanine-pNa or N-succinyl-L-alanyl-Lalanyl-L-prolyl-L-leucine-pNa, Sigma) the mixture was incubated for 30 min at 37° C. It was established that the reaction rate was linear. All solutions were made in 0.1 M Tris-HCl buffer (except for elastase: 0.2 M Tris-HCl) pH 7.9, containing 0.02 M CaCl₂. The reaction was stopped with 0.15 ml 30% acetic acid and the absorption was measured at 405 nm. One unit of enzyme activity was defined as 1 pmol of released p-nitroanilide per min at 37° C. All experiments were made in triple. Each fraction was tested for inhibition of the total proteolytic activity present in ileostomy effluent with azocasein as substrate (see above). Proteolytic activity was defined as mg hydrolyzed azocasein per 60 min. All inhibitor activities were expressed as suppressed protease activity per mg protein.

Protein Determination

In each of the 126 fractions protein concentration was determined using the Pierce BCA-200 Protein Assay Kit (see above). Electrophoresis of the fractions was performed in 20% polyacrylamide gel (Bio-Rad, Hercules, Calif.) in the presence of 0.1% sodium dodecyl sulfate and with and without 6-mercaptoethanol (SDS-PAGE). Seven pre-stained protein markers were used and ranged from 237 kD (myosin) to 7.2 kD (aprotinin). Gels were stained with 0.1% Coomasssie brilliant blue R-250 in 25% ethanol containing 5% formaldehyde

Skin Tests

Effect of Potato Proteins on the Development of Protease-Induced Dermatitis

Propylene chambers of 1 cm fitted with a pad of 10 by 10 mm were filled in duplicate with 50 μl of a test solution immediately prior to application. After application on the skin of the upper part of the back, the chambers were secured in position with paper adhesive tape; the distance between the test chambers was 15 mm. One series of 5 test chambers was placed from cranial to caudal, a second identical series from caudal to cranial. The composition of the test solutions was as follows:

A. Mixture of equal amounts of pancreatic elastase, trypsin and α-chymotrypsin (Sigma; for details see: Characterization of potato inhibitor fraction 2) dissolved in sterilized (by steam sterilization, 15 min at 121° C.) supernatant of ileostomy effluent; end concentration of the enzyme mixture was 1% (w/v).

C. Test solution A with potato proteins (fraction 2) in a concentration of 1% (w/v.

Control solutions were composed as follows:

B. Potato proteins (fraction 2), 1% (w/v) in sterilized supernatant of ileostomy effluent.

D. Sterilized supernatant of ileostomy effluent.

E. Potato proteins 1% (w/v) in PBS.

The pH of each test solution was 7.0. All test solutions were fresh prepared immediately prior to administration. After 24 hours of occlusion the test chambers were removed and the skin was rinsed with tap water. The test sites were inspected for erythema and dermatitis after 1, 2, 4, 6 and 24 hours according to a dermatitis severity scale [Patil S M., Patrick E, Maibach H I: Animal, human, and in vitro test methods for predicting skin irritation. Dermontotoxicology 1996;30:411-430 ed. Marzulli F N, Maibach H I 5° ed. (Taylor and Francis USA and UK)], summarized in table 8. Examination was done in a “blinded’ manner by the same investigator. Four and 24 hours after removal of the test chambers a photograph of the involved area was taken.

TABLE 8
Visual analog scoring system (VAS) for erythema and dermatitis,
after Patil et al., supra.

| | |
|---|---|
| 0 | Negative normal skin |
| ± | Questionable erythema not covering the entire area |
| 1 | Definite but slight erythema |
| 2 | Well defined erythema with slight oedema |
| 3 | Erythema with oedema and papulas and vesiculas |
| 4 | Severe erythema and erosions (integrity of skin is affected) |

Applicability of Potato Proteins in a Cream

Potato proteins (fraction 2) were mixed with a neutral cream (based on decyloleate, 60% water), 1% (w/w). One gram of the cream with potato proteins and one gram of the control cream (with no potato proteins) were placed, in duplicate, respectively at the left and the right upper part of the back. Thereafter test chambers with 50 μl test solution A (the mixture of pancreatic proteases dissolved in sterilized supernatant of ileostomy effluent, were placed at the skin that previous had been treated with cream. After 24 hours the test chambers were removed and the test sites were examined as described above.

Allergic Reactions

Potato protein fraction 2 (1% in phosphate buffer, w/v) was tested for induction of allergic contact dermatitis with the patch test at the back of sixty-three patients suffering from contact dermatitis, as 1 of 25 other potential allergens. After 48 and 72 hours of occlusion the response was evaluated.

Thirteen patients suffering from type I food allergic reactions were challenged by skin prick test with fraction 2 potato proteins (1% in phosphate buffer pH 7.4, w/v). One of the patients had complaints during contact with raw potatoes and had a previous positive skin reaction after challenge with extract from raw potatoes.

Development of Peri-Anal Dermatitis after IAA

After operation for IAA, the anal area of the patients was inspected daily, during hospitalization, for the development of dermatitis using a visual analog scoring system (VAS) according to Patil et al, supra, see table 1. Furthermore the patients completed a pain form, ranging from 0 (no pain) to 10 (extremely painful) as subjective observation.

Statistics

The Mann-Whitney U test was used to compare the proteolytic activity in feces from patients and healthy subjects. To compare the effect of protease inhibitors in the skin test, the sign-test was used. The coefficient of correlation (r) was calculated based on the least-squares criterion.

Results

Proteolytic Activity in Feces of Patients and Healthy Subjects

To assess the amount of proteases in feces, proteolytic activity of both healthy subjects and patients with intestinal resections was assayed. Table 9 shows that the total protease activity in feces from patients with intestinal resections of colon and/or ileum was significantly higher than in feces from healthy subjects. In fecal samples from 5 healthy subjects no activity at all was determined. The water content of the feces from healthy persons is relatively low (about 70%) and each fecal sample was formed. Feces from the patients were less formed, contained less dry material and were often watery (diarrhoea).

TABLE 9
Proteolytic activity in feces of healthy subjects and patients with
intestinal disorders

| | | | Feces | | |
|---|---|---|---|---|---|
| | | Proteolytic activity* | | % Dry matter | |
| | | median | | Median | |
| | n | (range) | p^{†} | (range) | P^{†} |
| Healthy | 20 | 15.7 (<0.1-64.4) | | 30.1 (16.5-30.3) | |
| subjects | | | | | |
| Patients: | | | | | |
| Ileostomy | 8 | 291.2 (49.6-754.3) | <0.01 | 8.5 (4.2-12.0) | <0.01 |
| Ileum | 14 | 112.0 (58.8-188.0) | <0.01 | 7.2 (3.8-11.3) | <0.01 |
| reservoir | | | | | |
| (pouch) | | | | | |
| Crohn's | 14 | 250.4 (144-488.4) | <0.01 | 13.6 (8.4-17.5) | 0.01 |
| disease | | | | | |
| (with | | | | | |
| resections) | | | | | |

*Expressed as mg hydrolyzed azocasein/h/g feces; ^{†} Value of difference versus healthy subjects (Mann-Whitney U test)

Peri-anal dermatitis of patients with intestinal disorders resembles diaper rash and pancreatic proteases are probably the main cause of these skin irritations; therefore proteolytic activity was estimated in feces from infants and children up to 8 years. FIG. 8 shows that protease activity in feces from infants up to one year old was very high and age dependent (r, after reciprocal transformation=0.54, p<0.01). Infants up to 12 months showed an increased fecal water content (median percentage dry material was 18.5, ranging from 11.3-28.9%), compared to children of 12-24 months (28.8, range 12.3-82.9), children of 2-6 years (26.9, range 19.9-36.1) and healthy adults (30.1, range 16.5-40.8); p<0.01.

Fecal Proteolytic Activity and the Development of Dermatitis after IAA

To investigate the possible relation between the development of peri-anal dermatitis after IAA, and fecal proteases, the anal area of these patients was examined daily according to VAS and the focal proteolytic activity and pH were determined. Proteolytic activity in feces from patients after IAA was found to be low during the first 6 days after the operation, (median 3.4, range 0-62 mg hydrolyzed azocasein/h but increased gradually in the next 10 days to median 87.6 (range 14.3-29.5) mg hydrolyzed azocasein/h during day 10 to 14 (see FIG. 9). FIG. 10 shows that in the first week after the operation the pH of the feces was high (median 8.5, range 7.6-9.4). During the next week a decrease (to median 6.9) was observed, but the pH showed large fluctuations.

During the first 4-5 days after the operation, when the feces is very watery, the patients are supplied with a drain. In this period only 3 of 48 patients developed a (slight) peri-anal dermatitis. After removal of the drain the peri-anal skin is in close contact with the feces as a result of incontinence and the high frequency of defecation. FIG. 11 shows that 96% of the patients developed a slight (27%, score±and 1), moderate (21%, score 2) or severe dermatitis (48%, score 3-4) in the peri-anal area during their stay in the hospital. Only 2 patients did not have dermatitis at all; one of them was completely continent immediately after removal of the drain. The (subjective) pain score was largely in line with the severity of the dermatitis. Each of the 46 patients who developed peri-anal dermatitis still suffered from this injury when leaving the hospital.

Feces from the patients after IAA was extremely watery during the first 14 days after the operation: the total amount of fecal effluent was very high, about 2.5-4 liter daily with 2.8 (range 2.1-3.5) % dry matter. Consequently the daily amount of proteases present in feces of IAA patients, from day 6 after surgery, is comparable with that of patients with a normally functioning ileum reservoir. The protease activity as shown in Table 9 is expressed per gram wet feces.

Inhibition of Proteolytic Activity in Feces by Potato Protein Fractions

To establish their capacity to suppress fecal proteolytic activity, increasing amounts of potato proteins were mixed with feces from patients with different intestinal diseases and feces from an infant. A crude (fraction 1) and a more purified protein fraction (fraction 2) with protease inhibitor activity were prepared from potatoes, variety Bintjes. This variety is available during the whole year. Both fractions were tested with feces from subjects with a high proteolytic activity, patients with an ileostomy, patients with an ileum reservoir, patients after IAA and infants of four months old. As shown in FIG. 12, both fractions were able to inhibit the majority of fecal protease activity, however fraction 2 was more effective. Fraction 2, in a concentration of 5%, inhibited the protease activity in feces from an IAA patient completely (100%); in a 10% concentration, 94% of the very high proteolytic activity in baby feces was inhibited. Furthermore a dose-response reaction was found. Inhibition of proteases was never found to be reversible.

Inhibition of Elastase Activity from Mouse Macrophages by Potato Proteins

To determine suppression of leucocyte elastase, potato proteins (fraction 2) were added to lysate of activated mouse macrophages and to purified human leucocyte elastase. Proteolytic activity was very low in macrophages growing in cell culture medium. However after stimulation with TNF-α the cells produced a considerable amount of elastase-like enzymes. These proteases did not degrade azocasein, but were demonstrable with SAAPLPNA as the substrate. When potato protein fraction 2 in a concentration of 1% was added, 80% of the protease activity was inhibited and with 20% potato protein hardly any activity was left (see FIG. 13). Also commercial purified human leucocyte elastase (Sigma) with an activity of 1.39±0.19 U on SAAPLPNA was effectively inhibited by (1%) potato protein fraction 2 to 0.20±0.09 U.

Characterization of Protease Inhibitor Fraction 2

Separation by gel chromatography of potato protein fraction 2 resulted in three protein peaks, A, B and C (FIG. 14a). SDS-PAGE electrophoresis of the fractions from peak A revealed two different bands; a major band was found at 50 kDa and a minor at 6.5 kDa. None of the tested proteases was inhibited, indicating that no inhibitors of trypsin, α-chymotrypsin and elastase were present in fraction A. In the fractions between peak A and B the 50 kDa band was still present, and a minor band at 40 kDa. Just before peak B a 25 kDa band was present, corresponding with strong inhibition of α-chymotrypsin, indicating the presence of α-chymotrypsin inhibitors.

FIGS. 14b and c show that the strongest inhibition of trypsin activity completely corresponded with peak B, but also in the protein fractions between peak B and C considerable inhibition of activity was found; elastase and α-chymotrypsin inhibition assays indicated the presence of inhibitors of these enzymes in fractions between protein peak B and C. Inhibition patterns of fecal proteolytic activity were similar to those of elastase and α-chymotrypsin. SDS-PAGE electrophoresis of the fractions from peak B showed at least three different major protein bands between 25 and 20 kDa and a band at 17 kDa. In the fractions between peak B and C 2 main bands at 22 and 17 kDa were present. No protein bands and no inhibition of protease activity were observed in peak C. No differences in protein bands were found on SDS-PAGE with and without β-mercaptoethanol.

Skin Tests

Effect of Potato Proteins on the Development of Protease-Induced Dermatitis

The hypothesis that potato proteins can prevent protease-induced skin irritation was assessed by applying test chambers, filled with a protease solution (solution A) and a mixture of proteases and potato proteins (solution C), to the skin of human volunteers for 24 h.

The exact compositions of the test- and the control solutions that were used for the skin tests are summarized in table 10. In this table it is shown that the protease activity measured in test solution A (proteases in sterilized ileostomy effluent) is almost completely inhibited by the potato protein fraction 2 (solution C). During in vitro incubation at 37° C. the activity of the enzyme mixture decreased. The activity of trypsin, α-chymotrypsin and elastase, used to compose the enzyme mixture and shown in table 10, demonstrates that each of these proteases degrades azocasein.

TABLE 10
Composition and proteolytic activity of test and control solutions
used for skin test

| | Test solutions | | Control solutions | | |
|---|---|---|---|---|---|
| | A | C | B | D | E |
| Sterilized ileostomy | + | + | + | + | − |
| effluent | | | | | |
| PBS | − | − | − | − | + |

Supplements:

| | | | | | |
|---|---|---|---|---|---|
| Protease mixture, | + | + | − | − | − |
| 10 mg/ml* | | | | | |
| Potato proteins, | − | + | + | − | + |
| 10 mg/ml^{†} | | | | | |

Proteolytic activity^{‡}:

| | | | | | |
|---|---|---|---|---|---|
| At the start of the | 1623.1 ± 73.7 | 9.0 ± 0.7 | n.d. | n.d. | n.d. |
| skin test | | | | | |
| After 5 h of incubation | 840.0 ± 52.1 | n.d. | n.d. | n.d. | n.d. |
| at 37° C. | | | | | |

Activity of the individual proteases*:

| | |
|---|---|
| Trypsin | 1318.3 ± 56.8 |
| α-Chymotrypsin | 356.3 ± 35.6 |
| Elastase | 278.6 ± 15.4 |

*Equal amounts of pancreatic trypsin, α-chymotrypsin and elastase;
^{†}fraction 2;
^{‡}expressed as mg hydrolyzed azocasein/h/ml;
+ = present in the solution;
− = not present in the solution;
n.d. = no detection of activity

Table 11 and FIG. 15 show that pancreatic enzymes, dissolved in sterilized ileostomy effluent (test solution A), cause a severe skin damage at the back of healthy volunteers within 24 h. This was completely prevented by addition of potato proteins to this solution.

TABLE 11
Skin irritation after 1 and 24 hours exposure to pancreatic proteases

| 1 H after removal the chambers | | | | | |
|---|---|---|---|---|---|
| Severity score^{†} | | | | | |
| 0 | ± | 1 | 2 | 3 | 4 |

Test solutions

| | | | | | | |
|---|---|---|---|---|---|---|
| A. Protease mixture in IE^{‡} | 1§ | — | 2 | 1 | 1 | 15 |
| C. Protease mixture + potato. | 13 | 5 | 2 | — | — | — |
| Proteins in IE. | | | | | | |

Control solutions

| B. Potato proteins in IE. | 14 | 4 | 2 | — | — | — |
|---|---|---|---|---|---|---|
| D. IE. | 10 | 9 | 1 | — | — | — |
| E. Potato proteins in PBS | 19 | — | 1 | — | — | — |
| | 24 H after removal the chambers | | | | | |
| | Severity score^{†} | | | | | |
| | 0 | ± | 1 | 2 | 3 | 4 |

Test solutions

| | | | | | | |
|---|---|---|---|---|---|---|
| A. Protease mixture | 1 | 1 | 2 | 1 | 2 | 13 |
| in IE^{‡} | | | | | | |
| C. Protease mixture + potato. | 20^{a} | — | — | — | — | — |
| Proteins in IE. | | | | | | |

Control solutions

| | | | | | | |
|---|---|---|---|---|---|---|
| B. Potato proteins in IE. | 20 | — | — | — | — | — |
| D. IE. | 17 | 3 | — | — | — | — |
| E. Potato proteins in PBS | 20 | — | — | — | — | — |

*Value of difference versus test solution A (sign test);
^{†}Visual analog system: see table I;
^{‡}For detailed information about composition and properties of the solutions used in the skintest, see table III;
IE = (sterilized) ileostomy effluent;
§Numbers of test sites scoring positive;
^{a}p < 0.01; 10 subjects were tested in duplo.

One of the test persons did not show any skin irritation with solution A (score 1 and ±), one showed only slight erythema (score 1); the other 8 subjects developed moderate to severe dermatitis. Twenty-four hours after removal of the test chambers, erythema induced by the control solutions had disappeared, except for a slight reaction by solution D (supernatant of sterilized ileostomy effluent) for 3 subjects. A reaction to potato proteins (solution C and E) was not observed.

Applicability of Potato Proteins in A Cream

Processing potato proteins into a cream was found to be possible without loss of inhibitory capacity. No skin irritations were found when the skin was treated with the potato protein containing cream, prior to application of the protease mixture. On the other hand, when the skin was treated with the control cream (without potato proteins), 7 of 8 test sites showed irritation (severity score 2-4).

Allergic Reactions

The possibility of adverse reactions to potato proteins was investigated by challenging subjects with a history of type I and type IV hypersensitivity. None of the 63 patients suffering from allergic contact dermatitis (type IV) who were challenged with fraction 2 potato proteins showed a skin reaction. Thirteen patients suffering from food allergic (type I) reactions, including the patient with complaints during contact with raw potatoes and a positive skin reaction after challenge with extract from raw potatoes, did not show any reaction to the skin prick test with fraction 2 potato proteins.

Discussion

The first part of our study revealed a very high frequency of irritant dermatitis after IAA. To the patients this complication is found to be the worst part of their stay in the hospital. Two factors determine the start of the dermatitis: removal of the drain from the anal canal, resulting in a close contact of the peri-anal skin with the watery feces and the increased fecal proteolytic activity. As shown, about six days after surgery the pH is, though decreasing, merely alkaline (between 8.5 and 6.0), still optimal for pancreatic proteases and resemble the situation in the proximal part of the ileum where dietary proteins are degraded to amino acids.

Our data showing strongly increased protease activity in fecal samples from patients with intestinal resections, are in line with earlier studies of our group and with studies of Layer et al [Am J Physiol 1986;251:G475-G480] for healthy subjects. The major proteolytic activity is derived from pancreatic trypsin, α-chymotrypsin and elastase, belonging to the class of serine proteases. It is not exactly known what happens to proteases that enter the duodenum as secretions of the pancreas. A part of their activity is lost in the terminal ileum, probably due to absorption and/or action of endogenous inhibitors. It is likely that the colon flora is responsible for further inactivation of the proteases. In feces of healthy subjects only a low enzyme activity can be estimated, which is largely of bacterial origin; bacterial proteases belong mainly to the class of metallo- and cysteine proteases. We may conclude that it is likely that in feces of patients with gastrointestinal disorders and infants, pancreatic proteases exceed bacterial proteolytic proteases 25 to 100 times.

Also the transit time in the colon is an important factor: with a fast rate of passage a larger part of the pancreatic proteases will persist and consequently will be present in feces. This is undoubtedly the situation in patients with intestinal resections. Soft or liquid stools, high frequency of defecation and soiling lead to close contact of the peri-anal skin with proteases and increase occurrence of dermatitis in this area. In idiopathic pruritis ani intermittent seepage from the anal canal is seen as the most important contributing factor; unactivated pancreatic proteases seem to be the injuring agents.

Treatment of peri-anal dermatitis is largely limited to conventional applications, such as greasy ointments often with zinc or aluminum compounds, making a barrier, and topical corticosteroids. More recently sucralfate, a protein binding basic aluminum salt of sucrose octasulfate; and cholestramine, a bile acid sequestrant, which irreversible can bind bile when applied topically, have been shown to reduce peri-stomal and peri-anal irritation caused by feces. In our study protease inhibitors were chosen to neutralize excess of pancreatic proteases in order to prevent skin injury. A second effect might be a reduction of inflammation by inhibiting cellular proteases and diminishing enzyme release and degranulation of polymorphonuclear leucocytes.

These protease inhibitors have to fulfil certain conditions. First they have to be non-toxic for humans, second they have to be capable to inhibit each of the major intestinal proteases, pancreatic trypsin, α-chymotrypsin and elastase. Several potential non-toxic purified protease inhibitors, most of them from bacterial or vegetal origin, such as Streptomyces-, soy bean-, lima bean-, corn- and potato inhibitors are known, and commercially available. In general each of these purified inhibitors has a narrow range of action and consequently combinations of inhibitors are required to inhibit the total intestinal protease activity. From the literature we know that potato tubers are an extraordinarily rich source of a variety of protease inhibitors, representing 25-30% of potato juice protein. These protease inhibitors have been biochemically identified and extensively characterized with respect to their function during the past thirty years. With gel chromatography of potato protein fraction 2 and SDS-PAGE of the eluates we confirmed the presence of active inhibitors of pancreatic proteases and their protein nature. We found at least 4 different inhibitors of elastase, trypsin and α-chymotrypsin with molecular weights between 25 and 20 kDa and 17 kDa and an α-chymotrypsine inhibitor of 25 kDa. This 25 kDa inhibitor of α-chymotrypsin resembles a Kunitz-type protease inhibitor with a high affinity for chymotrypsin and a low inhibiting activity against trypsin. Consistent with our findings, inhibitors of trypsin and α-chymotrypsin with molecular weights ranging from 25-20 kDa, acting against both enzymes and belonging to the group of serins proteases, have been isolated from potato protein and described. Pouvreau et al [J Agric Food Chem 2001;49: 2864-2874] characterized 20-22 kDa proteins able to inhibit elastase as well as trypsin and α-chymotrypsin; this was in line by our results. Furthermore we determined elastase, trypsin and α-chymotrypsin inhibition by proteins with a molecular weight of 25 kDa; as far as we know no literature describing 25 kDa elastase inhibitors is available. The inhibition pattern of elastase in the eluates after gel chromatography showing two peaks, confirms the presence of (at least) 2 different elastase inhibitors.

According to its inhibiting activity against trypsin and α-chymotrypsin the 17 kDa protein we detected can be identified as the inhibitor described by Revina et al [Biochemistry (Moskow) 1995;60:1411-1416]; this protein has two independent active centers for one trypsin molecule and one chymotrypsin molecule and interacts with these enzymes in a 1:1 molar ratio.

The major bands from peak A representing proteins with a molecular weight of 40 kDa, can be attributed to heat-resistant potato lectin. The minor protein band at 6.5 kDa from these fractions did not show protease inhibiting capacities to elastase, trypsin or α-chymotrypsine; this band might represent a carboxypeptidase inhibitor as described by Ryan et al [J Biol Chem 1974;17:5495-5499].

The pre-treatment of potato protein fraction 2 by ascorbic acid and heating (15 min at 65° C.) might separate proteins in subunits and is probably the cause that reducing and non-reducing SDS-PAGE electrophoresis showed bands at the same molecular weight. Another effect of this treatment was the removal of the major part of patatine, the storage protein of potatoes, resulting in minor bands of 40 kDa in protein fraction between peak A and B. Peak C contained no inhibitors and is probably composed of polyphenols; we did not determine a peak consisting of oxidized polyphenols (described as fraction IV by Pouvreau et al [J Agric Food Chem 2001;49: 2864-2874]) since oxidation was prevented by addition of ascorbic acid to the raw potato juice.

The results obtained are clearly dependant on the degree of purity of the inhibitor fraction. The inhibition was directly proportional to protein concentration and the initial proteolytic activity of the feces, leading to maximal blocking of 100% of the activity in feces from patients with intestinal resections and 94% of the activity in feces from infants. The age dependency of proteolytic activity in feces from infants is probably a reflection of immaturity of the intestinal functions. The development seems to be slow and to be determined by both endogenous and environmental factors such as pancreatic secretions, transit time, establishment of the micro-flora and diet. The high level of proteases in infant feces is probably the major cause of diaper dermatitis. Although diaper rash resembles peri-anal dermatitis in patients with gastrointestinal resections, the etiology is more complicated. In the diaper urea from urine is converted to ammonia by urease produced by skin or fecal bacteria, which results in a rise of pH. Using hairless mice Berg et al [Pediatr Dermatol 1986;3:102-106] found increased skin irritation by proteases when pH of the test buffer was more alkaline. At pH 6.8 and 7.8 human fecal protease activity was found to be respectively 3 and 4 times higher than at pH 5.2. Increase in pH is significantly associated with elevated frequency of diaper dermatitis.

To investigate the effect of protease inhibitors in preventing skin damage by pancreatic proteases a human skin irritation assay was designed. It is too risky to apply fresh ileal output to the human skin. Therefore a “natural” environment was created by using sterilized supernatant from ileostomy effluent to dissolve purified proteases. Intestinal components, which might influence the development of dermatitis, such as bacterial antigens, bile acids, mucus glycoproteins etc, are still present in the test solution and might have an additional effect. Pancreatic enzymes in feces keep their activity for months, but purified enzymes are less stable. Therefore at the start of the skin tests the protease activity in the test solution had to be at least 3 times higher than in fresh feces of patients and infants, because activity decreased during the test to about physiological values of feces of these groups. A relative high protease activity at the start of the test had the advantage of a fast development of skin irritation. Anderson et al [Contact dermatitis 1994;30:152-158] reported visual skin irritation from day 5 of occlusive exposure to proteases (half the concentration we used in our experiments) in buffer solution; the lack of fecal components in the test solution might be the cause of the slow development of the dermatitis, fecal components, such as pancreatic lipase and bile acids, have been suggested to play a role in the development of dermatitis by removing ‘the protective lipid layer’. However, the skin test in this study shows that lipase is not essential for this process and that bile acids, which are still present in the sterilized fecal supernatant that was used, play a minor role; 3 of 20 test sites with only sterilized supernatant of ileostomy effluent (solution D) showed a very slight (questionable) erythema. Exposure to proteases induced moderate to severe skin irritation to 8 of 10 subjects, except for 2 subjects. One developed slight erythema, but the second subject did not show any irritation at all at both test sites. This suggests high inter-individual variation in epidermal barrier function towards pancreatic proteases. Also treatment of the skin with a cream containing potato proteins, prior to application of the protease mixture completely prevented skin irritation. Furthermore no adverse effects at all were observed. Sensitivity to potatoes is fairly uncommon, in contrast to other foods. Consumption of potatoes for food or peeling of raw potatoes may elicit type-I allergic reactions probably due to patatin, the main storage protein of potato tubers. Type IV contact dermatitis, caused by potatoes is rarely reported. Although the number of patients we tested for allergic reactions to potato proteins is small and needs to be increased, the results are encouraging.

In the epidermis is a need for continuous renewal and degradation of intracellular contacts; proteases that are responsible for degradation of cohesive structures in the skin are stratum corneum chymotryptic enzyme (SCCE) and stratum corneum tryptic enzyme (SCTE). This process is tightly controlled by several factors among which binding to specific inhibitors, such as locally produced elafin (also known as skin-derived antileucoproteinase) and secretory leukocyte proteinase inhibitor (SLPI). The balance between protease inhibitors and proteases determines the local proteolytic activity. During inflammation the balance might be disturbed by excessive neutrophyl elastase release, resulting in cell and tissue damage. To control neutrophyl elastase in chronic inflammation protease inhibitors are seen as attractive potential therapeutic agents.

In a small study treatment of atopic dermatitis with protease inhibitors alpha₁-proteinase inhibitor was found to have a wound healing effect on therapy-resistant atopic dermatitis. Wiedow et al [Dermatol 1992;99:306-309] showed in vitro an inhibitotory effect of alpha₁-proteinase inhibitor and soy bean trypsin inhibitor on lesional elastase activity in psoriasis, contact dermatitis and atopic dermatitis. This is in line with our pilot study that showed that potato proteins suppress proteolytic activity released by activated macrophages. Consequently potato proteins might be beneficial to patients with skin inflammation.

These experiments demonstrate that potato protein fractions are capable to inhibit the larger part of the high proteolytic activity in feces from patients with gastro-intestinal resections and infants (in vitro) and prevented experimental protease induced dermatitis.

Protocol for the Purification of Protease Inhibitors from Potato, Variety ‘Bintje’

1. peel potatoes thick (removing glycoalkaloids) and wash thoroughly

2. grind in braunshaker

3. filter through cambric gauze

4. add 0.2% (w/v) ascorbic acid to filtrate (inhibition of oxidation-brown coloring)

5. centrifuge 30 min at 13.000 rpm (Sorval)

6. heat supernatant 15 min at 65° C. (waterbath) (inactivation polyphenoloxidase, to prevent brown coloring)

7. cool

8. repeat step 5

9. filter supernatant through paper

10. freeze filtrate and freeze-dry

11. make a 20% solution (w/v) in ice cold distilled water

12. keep solution on ice

13. add to the solution 1.6 parts ice cold ethanol (end concentration is 60%)

14. mix carefully for 5 min (precipitation of lectines)

15. centrifuge 10 min at 10.000 rpm (Sorvall) (removal of the lectines)

16. evaporate alcohol from supernatant with cold air

17.mix precipitate with distilled water and dialyse (8.5 kD) against excess of water

18. centrifuge dialysate 10 min 10.000 rpm (Sorvall)

19. freeze supernatant and freeze-dry

20. dissolve the powder (end concentration 10%) in glycerol gel with MOB (methylparahydroxybenzoate) as preservative

### FIGURES

FIG. 1: Inhibition of fecal proteolytic activity by products from potato juice.

Feces from 1 patient with a well functioning pouch was used.

Feces was used undiluted.

EURO's were used as 1:5, 1:10, 1:25, 1:50 and 1:100 dilutions in phosphate buffer pH 7.6.

Feces and EURO were mixed 1:1 for 10 minutes, then the mixture was diluted in phosphate buffer pH 7.6 1:12.5.

In both dilutions proteolytic activity was measured with azocaseine as substrate.

FIG. 2: Inhibition of fecal proteolytic activity by products from potato juice

Feces from 1 patient with an ileostomy was used.

Feces were used undiluted.

EURO's were used as 1:5, 1:10, 1:25, 1:50 and 1:100 dilutions in phosphate buffer pH 7.6.

Feces and EURO were mixed 1:1 for 10 minutes, then the mixture was diluted in phosphate buffer pH 7.6 1:12.5.

In both dilutions activity was measured with azocaseine as substrate.

FIG. 3: Inhibition of fecal proteolytic activity by products from potato juice

Feces from 1 patient 14 days after colectomy was used.

Feces were used undiluted.

EURO's were used as 1:5, 1:10, 1:25, 1:50 and 1.100 dilutions in phosphate buffer pH 7.6.

Feces and EURO were mixed 1:1 for 10 minutes, then the mixture was diluted in phosphate buffer pH 7.6 1:12.5.

In both dilutions proteolytic activity was measured with azocaseine as substrate.

FIG. 4 and FIG. 5: Inhibition of fecal proteolytic activity by products from potato juice Feces from 2 babies aged 4 months were used.

Feces were used diluted 1:1 in phosphate buffer pH 7.6 and centrifuged 10 minutes at 10,000 g

EURO's were used as 1:5, 1:10, 1:25, 1:50 and 1.100 dilutions in phosphate buffer pH 7.6.

Feces and EURO were mixed 1:1 for 10 minutes, then the mixture was diluted in phosphate buffer pH 7.6 1:12.5.

In both dilutions proteolytic activity was measured with azocaseine as substrate.

FIG. 6: Patch Test Chambers (van der Bend) of 10 by 10 mm, filed with 50 μl of a test solution were placed on the skin of the upper part of the back of 2 healthy subjects and fixed with Fixomull Stretch self adhesive tape; the distance between them was 15 mm. One series of 4 testchambers was placed from cranial to caudal, a second series from caudal to cranial.

The test solutions had the following composition:

A. elastase, trypsin and α-chymotrypsin, end concentration of each of the enzymes 1% (Enzyme Mix) soluted in sterilized fecal supernatant from an ileostomy patient (FS)

B. FS

C. Euro 2 (end concentration 5%) soluted in FS with Enzyme Mix

D. Euro 2 in FS

After 24 hours the test chambers were removed and the skin was rinsed with tap water. Sites were inspected for erythema and dermatitis after 1, 2, 4, 6 and 24 hours.

FIG. 7: The same patchtest as described under FIG. 6, but the crude inhibitor fraction was replaced by the more purified fraction (EURO 3).

E. EURO 3 in distilled water.

FIG. 8: Proteolytic activity in feces from healthy children. Proteolytic activity was expressed as mg azocasein hydrolyzed during 1 h per g feces.

FIG. 9: Fecal proteolytic activity of patients after ileoanal anastomosis. Proteolytic activity was expressed as mg azocasein hydrolyzed during 1 h per g feces.

FIG. 10: Fecal pH of patients after ileoanal anastomosis.

FIG. 11: Development of peri-anal dermatitis within 10 days after ileoanal anastomosis. Results of the examination were summarized according to the scoring system of Patil et al [6] (see Table 1).

FIG. 12: Inhibition of proteolytic activity in feces from patients with intestinal disorders and a healthy infant, by potato proteins. A: Potato protein fraction 1; B: Potato protein fraction 2.

FIG. 13: Inhibition of elastase activity from activated macrophages by potato proteins (fraction 2). Enzyme activity was expressed as U per ml, which is corresponding to the lysate of 3.10⁸ cells.

FIG. 14: Protein concentration (a) and protease inhibition after fractionation of potato protein fraction 2 on Superdex 75 column (b and c).

FIG. 15: Inhibition of protease induced skin irritation; back of a healthy volunteer 4 h after removal of the test chambers. At test site A a protease mixture (inducing skin irritation) was applied. At test site C the same protease mixture with potato proteins fraction 2, was applied; skin irritation was inhibited. At test sites B and D, control solutions were applied.

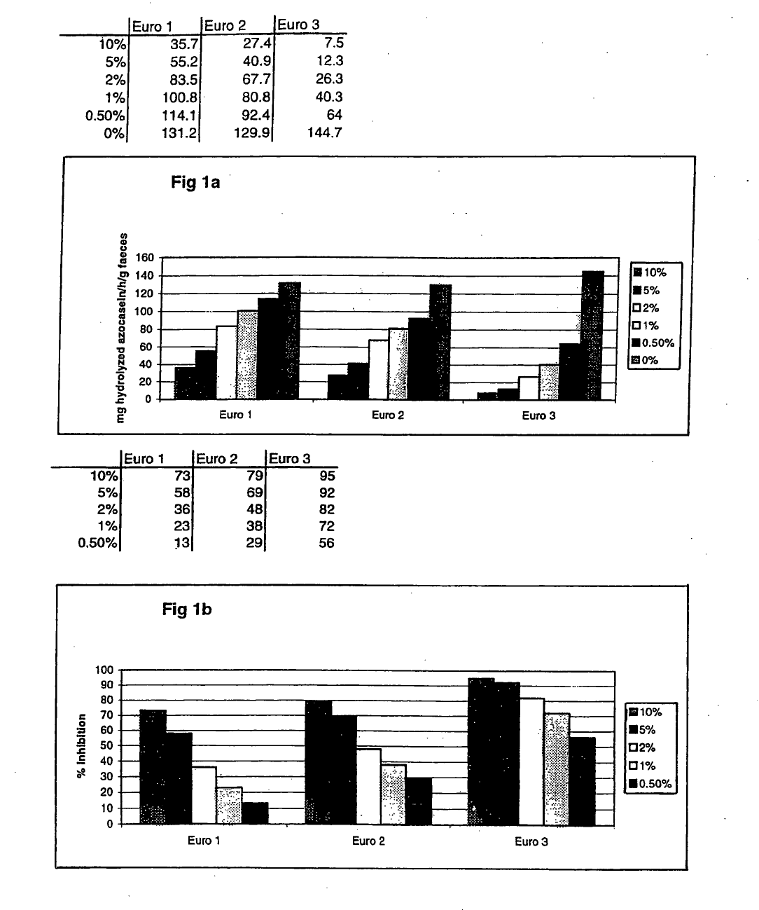

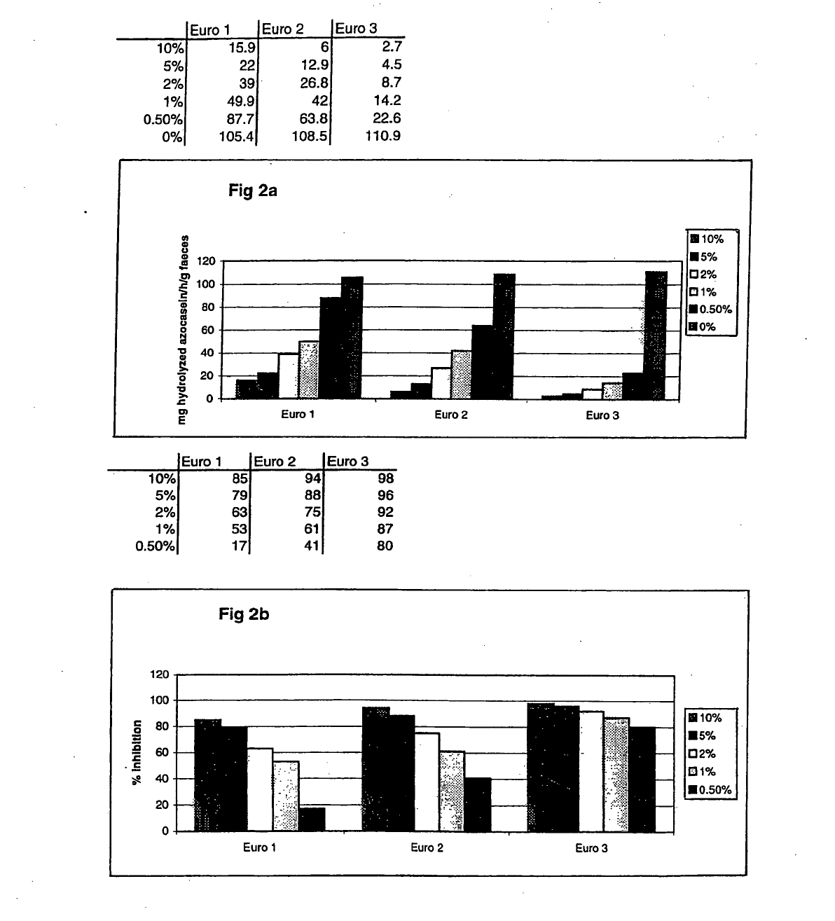

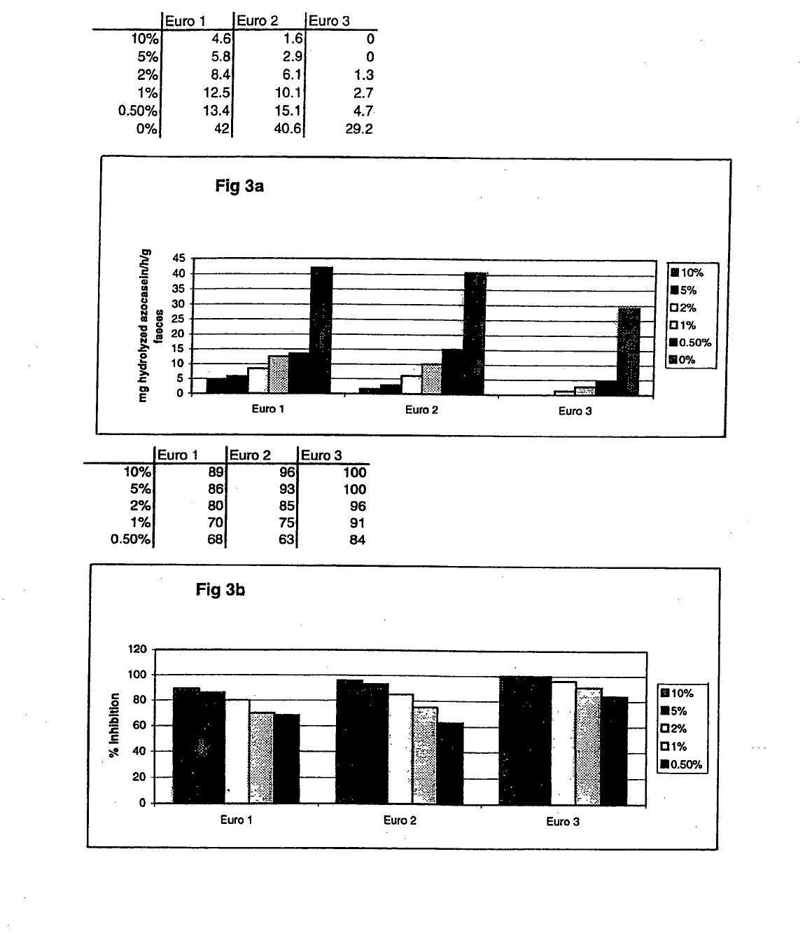

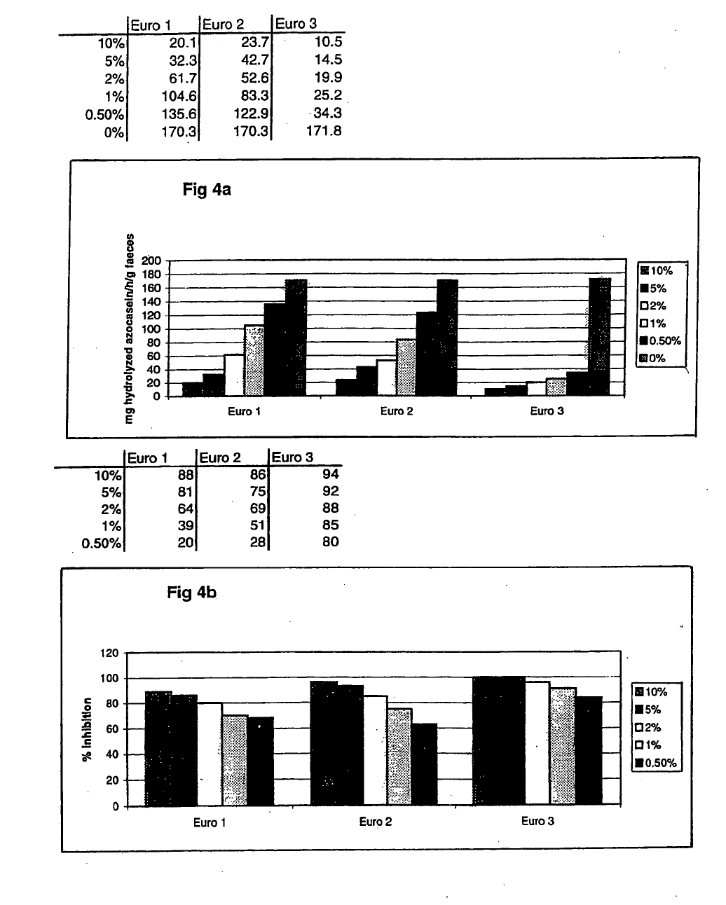

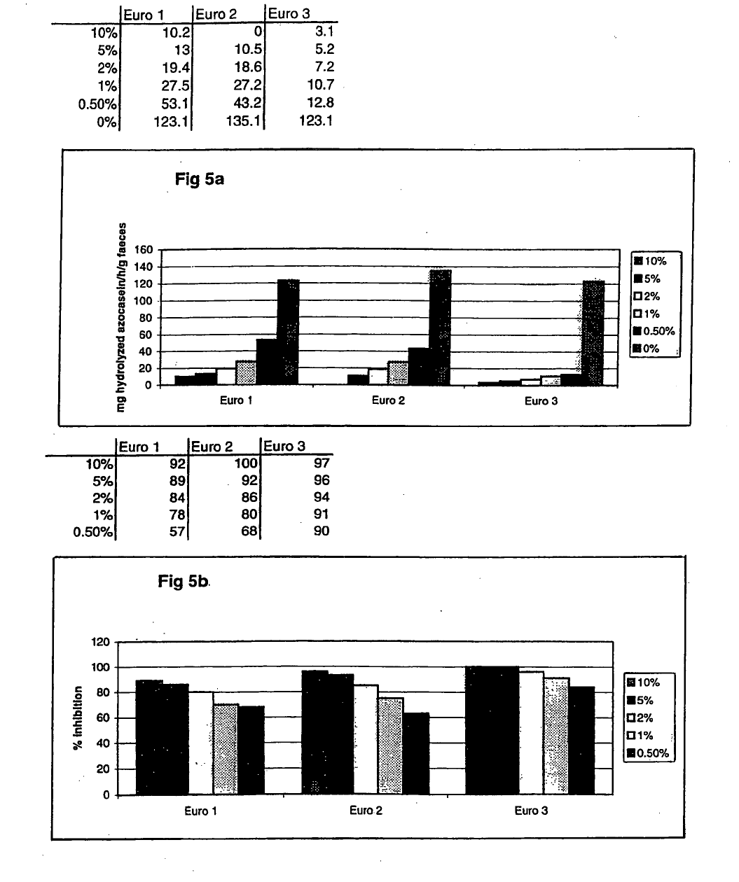

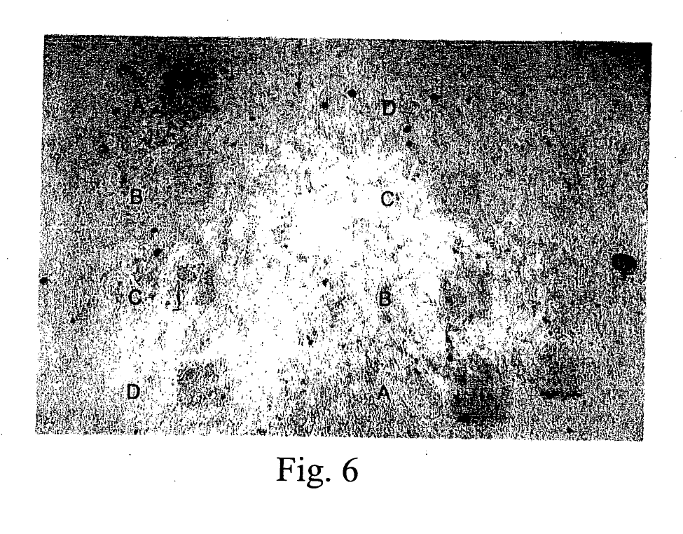

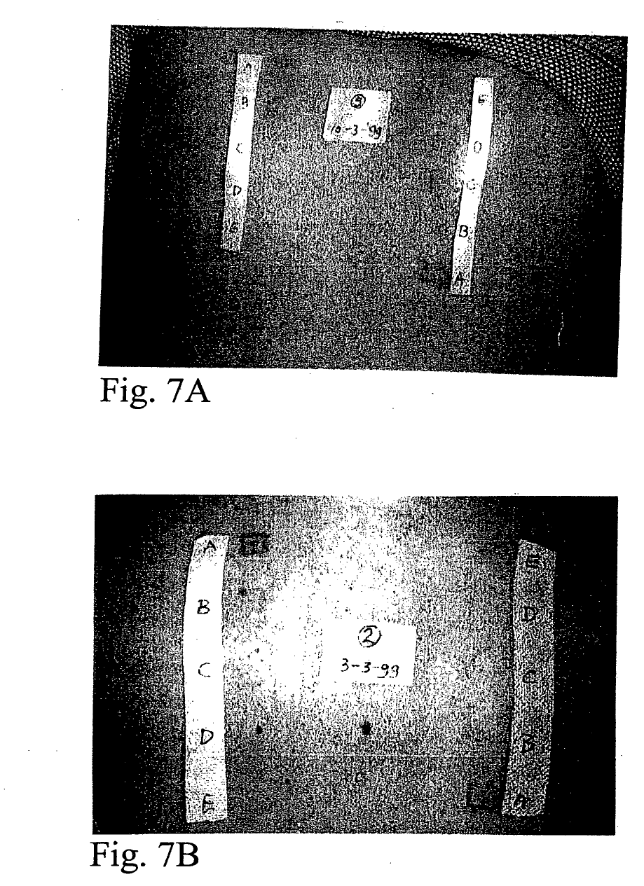

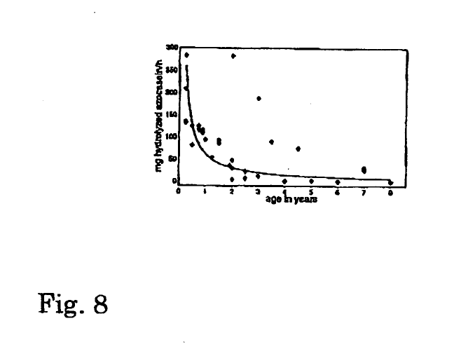

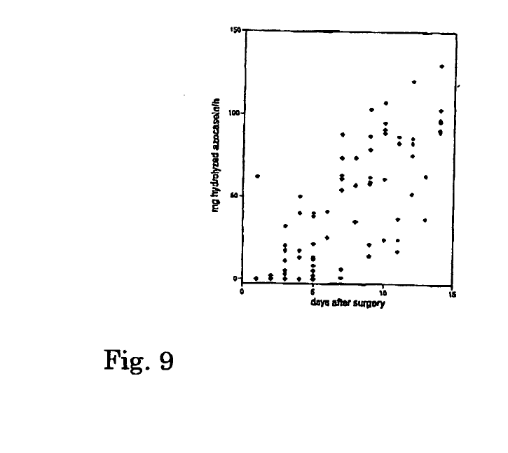

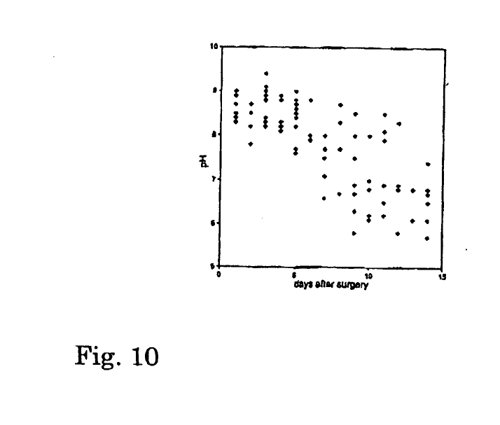

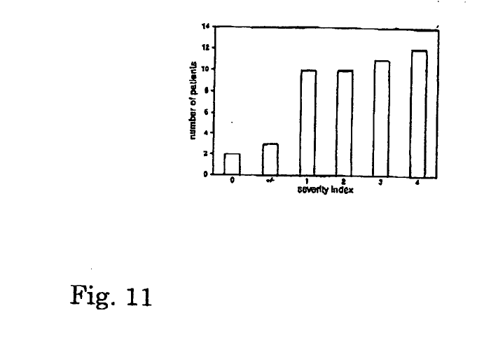

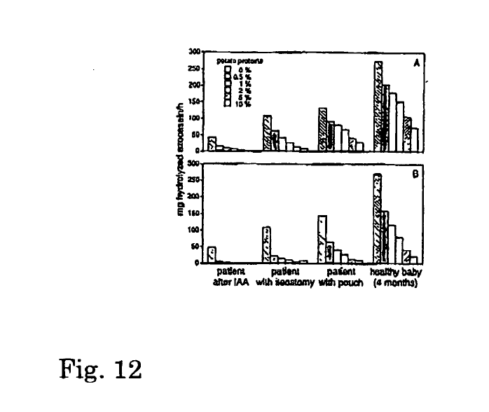

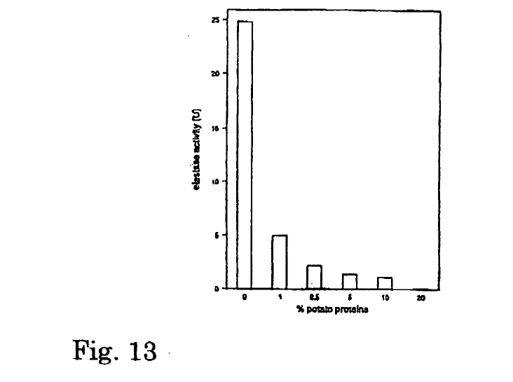

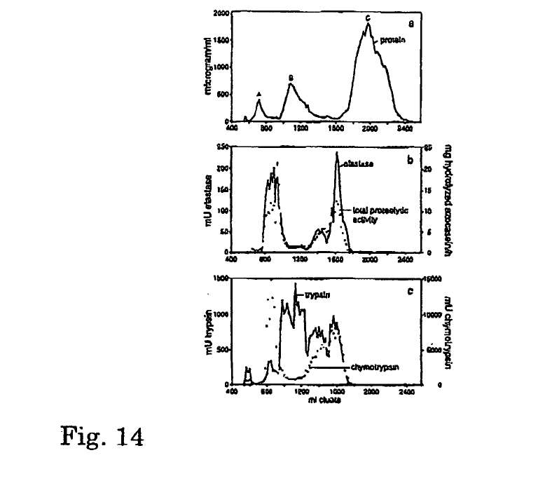

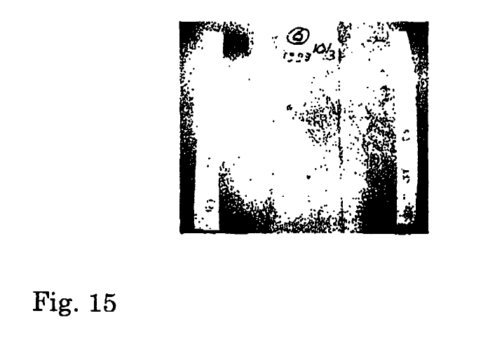

What is claimed is:

1. A skin care or topical pharmaceutical composition comprising at least one inhibitor capable of inhibiting proteolytic activity of a protease and a cosmetically or pharmaceutically acceptable vehicle, and wherein the inhibitor is derived from potato.

2. The skin care or topical pharmaceutical composition of claim 1, wherein said cosmetically or pharmaceutically acceptable vehicle is selected from the group consisting of aerosol spray, cream, dispersion, emulsion, foam, gel, lotion, mousse, ointment, pomade, powder, pump spray, solid, aqueous solution, hydro-alcoholic solution and stick.

3. The skin care or topical pharmaceutical composition of claim 1, comprising from 1 to 20 wt. % of the inhibitor based on the total weight of the composition.

4. The skin care or topical pharmaceutical composition of claim 1, having a pH in the range of about 4.8 to about 5.5.

5 The skin care or topical pharmaceutical composition of claim 1, further comprising cosmetic adjuncts, pharmaceutical adjuvants or supplements.

6. The skin care or topical pharmaceutical composition of claim 1 in the form of an aqueous liquid or hydrogel.

7. The skin care or topical pharmaceutical composition of claim 1 comprised in a band-Said, diaper, patch, towelette or wipe.

8. The skin care or topical pharmaceutical composition of claim 1 in the form of a lotion that can be absorbed into a bandage, diaper, patch, towelette or wet wipe base sheet.

9. The skin care or topical pharmaceutical composition of claim 1 in the form of a hydrogel comprising:
1 to 10 wt. % of a protease inhibitor from potato;
10-20 wt. % of one or more emollients;
1-5 wt. % of one or more thickeners;
0.1-0.5 wt. % of one or more preservatives; and
water to balance.

10. The skin care or topical pharmaceutical composition of claim 1 in the form of an aqueous liquid comprising:
1 to 10 wt. %. of a protease inhibitor from potato;
1-20 wt. % of one or more emollients;
0.5-3 wt. % of one or more thickeners;
0.1- wt. % of one or more preservatives; and
water to balance.

11. The skin care or topical pharmaceutical composition of claim 1 in the form of a rinsing fluid comprising:
1 to 10 wt. %. of a protease inhibitor from potato;
0.8-1.0 wt. % one or more buffers;
0.1-0.5 wt. % of one or more thickeners; and
water to balance.

12. The skin care or topical pharmaceutical composition of claim 1 in the form of a Lanette cream having a water content of more than 50%.

13. The skin care or topical pharmaceutical composition of claim 1 in the form of a “leave-on” product.

14. An article for topical administration of a skin care or topical pharmaceutical composition, said article comprising:
the skin care or topical pharmaceutical composition of claim 1.

15. The article of claim 14, wherein the article is selected from the group consisting of bandage, diaper, patch, towelette, and wipe.

16. The article of claim 14, wherein the article is of the type having an absorbent portion, said absorbent portion comprising the skin care or topical pharmaceutical composition of claim 1.

17. The article of claim 16, wherein the article is a cloth or disposable diaper.

18. The article of claim 14, wherein the article is a wet wipe.

19. The article of claim 18, wherein the wet wipe is packed in a pop up dispenser.

20. A method of treating or preventing perineal, peri-anal or peristomal inflammation caused by proteolytic activity of feces in a subject, comprising:
administering, topically, an inhibitor of proteolytic activity derived from a potato to the perineal, peri-anal or peristomal area of the subject, in unit dosage form containing about 0.1 to about 50 g of said inhibitor of proteolytic activity per day.

21. The method according to claim 20, wherein the inhibitor of proteolytic activity is administered as an ointment, cream, gel, spray, rinsing fluid or powder.

22. The method according to claim 20, wherein a wipe is used to topically administer the inhibitor of proteolytic activity to said subject

23. A diagnostic method for studying the effect of an inhibitor of proteolytic activity on inflammatory action of feces, the diagnostic method comprising:
sterilizing feces;
adding at least one proteolytic enzyme to said sterilized feces,
placing a sample of said feces with proteolytic activity on the skin of a healthy subject; and
monitoring the healthy subject skin for inflammatory action.

24. The diagnostic method according to claim 23 wherein said feces is taken from an infant or a patient.

25. A cloth of woven material to which is chemically associated an inhibitor of proteolytic activity.

26. A method to derive protease inhibitors from plants or plant-parts, comprising:
a) grinding the plant or plant parts to a pulp;
b) separating the solids from the pulp to provide a juice of the plant or plant-part;
c) coagulating bulk proteins in said juice, preferably by acidifying and heating of the juice;
d) separating the coagulated proteins from the juice;
e) adjusting the pH to a value of the juice of about 4.0; and
f) isolating the protease inhibitors from the juice by precipitating the protease inhibitors with sodium polyphosphate.
